(19) United States
(12) Patent Application Publication
(10) Pub. No.: US 2024/0156389 A1
(43) Pub. Date: May 16, 2024

(54) HEALTH MONITORING SYSTEM AND METHOD

(71) Applicant: ORTHO BIOMED INC., Vaughan (CA)

(72) Inventors: Mansour Abolghasemian, North York (CA); Jamal Esmaelpoor, Richmond Hill (CA); Babak Vaseghi, Brampton (CA); Masoud Sadeghisheikhtabaghi, Vancouver (CA); Seyed Babak Zianamin, Coquitlam (CA)

(73) Assignee: Ortho Biomed Inc., Vaughan, ON (CA)

(21) Appl. No.: 18/550,968

(22) PCT Filed: Mar. 18, 2021
(86) PCT No.: PCT/IB2021/052256

§ 371 Date: Sep. 17, 2023

(51) Int. Cl. A61B 5/366; A61B 5/00; A61B 5/0205; A61B 5/08; A61B 5/1455; A61B 5/28; A61B 5/353; A61B 5/355; A61B 5/358; G06N 3/0464

(52) U.S. Cl. CPC A61B 5/366; A61B 5/02055; A61B 5/0816; A61B 5/0823; A61B 5/14552; A61B 5/28; A61B 5/353; A61B 5/355; A61B 5/358; A61B 5/725; A61B 5/7257; A61B 5/7267; G06N 3/0464; A61B 2562/0219

(57) ABSTRACT

A method for health monitoring of a subject. The method includes measuring each of a plurality of physiological parameters once per a respective time period of a plurality of time periods. Measuring each of the plurality of physiological parameters includes measuring a heart rate of the plurality of physiological parameters by installing a sensor package on a region at a right side of a chest of the subject Installing the sensor package on the region includes placing a pair of electrocardiography (ECG) electrodes and an accelerometer in the sensor package on the region which includes the anterior edge of the right serratus anterior muscle of the subject.

### TECHNICAL FIELD

The present disclosure generally relates to biomedical engineering, and particularly, to biomedical signal processing.

### BACKGROUND ART

Some situations like worldwide pandemic outbreaks may rapidly increase the number of patients, and long hospitalization durations can impose significant workload on healthcare systems. While some sufferers do require hospitalization, most do not. To support those at home, accurate data may be vital for healthcare monitoring. In addition healthy subjects may also be interested in healthcare monitoring systems. For instance, sportsmen may use healthcare monitoring systems to monitor their performance. Some people may be interested in taking their vital health indicators during daily lives.

Conventional healthcare monitoring systems either use customized hardware designed for specific monitoring applications or transfer data to a server with advanced processing capabilities to reach accurate results [U.S. Pat. Nos. 9,854,986, 10,314,489, and 10,554,756]. Although these systems may be suitable for advanced medical applications, the cost of such systems may limit a widespread application in daily life by ordinary people.

There is, therefore, a need for a cost-effective healthcare monitoring system that may be capable of providing accurate measurements of physiological parameters. There is also a need for a method for obtaining physiological parameters with an efficient computational cost that can be implemented on commercial processors.

### SUMMARY OF THE DISCLOSURE

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for health monitoring of a subject. An exemplary method may include measuring each of a plurality of physiological parameters of the subject once per a respective time period of a plurality of time periods. In an exemplary embodiment, measuring each of the plurality of physiological parameters may include measuring a heart rate of the plurality of physiological parameters by installing a sensor package on a region at a right side of a chest of the subject. In an exemplary embodiment, installing the sensor package on the region may include placing an electrocardiography (ECG) electrodes pair of the sensor package on the region and placing an accelerometer of the sensor package on the region. An exemplary region may include a right serratus anterior muscle of the subject. In an exemplary embodiment, placing the ECG electrodes pair may include placing a pair of biocompatible cohesive ECG electrodes about one inch apart on the region in a vertical orientation.

An exemplary method may further include acquiring an ECG signal of the subject by acquiring each ECG sample of the ECG signal utilizing the ECG electrodes pair, acquiring a motion signal of the subject by acquiring each motion sample of the motion signal simultaneously with acquiring a respective ECG sample of the ECG signal utilizing the accelerometer, calculating a short-time Fourier transform (STFT) of the ECG signal responsive to a magnitude of a respective motion sample of the motion signal being smaller than a predetermined motion threshold, extracting a plurality of P waves, a plurality of QRS complexes, and a plurality of T waves from the STFT by applying a long short-term memory (LSTM) neural network on the STFT, and estimating the heart rate by calculating a number of the plurality of QRS complexes in a given period of time.

In an exemplary embodiment, measuring each of the plurality of physiological parameters may further include measuring an Oxygen saturation level (SpO₂) of the plurality of physiological parameters by placing a photoplethysmography (PPG) sensor of the sensor package on the region and measuring the SpO₂ utilizing the PPG sensor.

In an exemplary embodiment, measuring each of the plurality of physiological parameters may further include estimating a respiratory rate of the plurality of physiological parameters by acquiring a PPG signal from the subject, extracting a respiratory component of the PPG signal responsive to a magnitude of a respective motion sample of the motion signal being smaller than the predetermined motion threshold, extracting a refined signal from the respiratory component by applying a band-pass filter on the respiratory component, and obtaining the respiratory rate by applying an adaptive lattice notch filter (ALNF) on the refined signal. In an exemplary embodiment, acquiring the PPG signal may include acquiring each PPG sample of the PPG signal utilizing the PPG sensor simultaneously with acquiring a respective ECG sample of the ECG signal. In an exemplary embodiment, extracting the respiratory component may include removing a cardiac component of the PPG signal utilizing a sequential harmonic infinite impulse response (IIR) notch filter based on the heart rate.

In an exemplary embodiment, installing the sensor package may further include moving the sensor package on the region simultaneously with acquiring the ECG signal and acquiring the PPG signal, calculating a plurality of quality factors simultaneously with moving the sensor package, obtaining a subset of the plurality of quality factors, sticking a skin attachment piece at an optimal location of a plurality of locations in the region, and installing the sensor package on the skin attachment piece. In an exemplary embodiment, each of the plurality of quality factors may be associated with a respective location of the plurality of locations. In an exemplary embodiment, each respective quality factor in the subset may include a value larger than a predetermined ratio of a largest quality factor of the plurality of quality factors An exemplary optimal location may be associated with a respective quality factor in the subset.

In an exemplary embodiment, calculating the plurality of quality factors may include measuring a first correlation between the ECG signal and a reference ECG signal of a plurality of reference ECG signals, measuring a second correlation between the PPG signal and a reference PPG signal of a plurality of reference PPG signals, and calculating a quality factor of the plurality of quality factors by averaging the first correlation and the second correlation.

In an exemplary embodiment, measuring each of the plurality of physiological parameters may further include estimating a systolic blood pressure of the plurality of physiological parameters and a diastolic blood pressure of the plurality of physiological parameters by segmenting the PPG signal to a plurality of PPG segments and applying an end-to-end neural network on the plurality of PPG segments. In an exemplary embodiment, segmenting the PPG signal to the plurality of PPG segments may include extracting each of the plurality of PPG segments from the PPG signal at a respective time interval. An exemplary time interval may correspond to a respective QRS complex of the plurality of QRS complexes.

In an exemplary embodiment, applying the end-to-end neural network on the plurality of PPG segments may include extracting a first filtered PPG feature set of a first plurality of filtered PPG feature sets from a PPG segment of the plurality of PPG segments by applying a first convolutional layer of the end-to-end neural network on the PPG segment, generating a first averaged PPG feature set of a first plurality of averaged PPG feature sets by applying a first average pooling layer of the end-to-end neural network on the first filtered PPG feature set, generating a second filtered PPG feature set of a second plurality of filtered PPG feature sets by applying a second convolutional layer of the end-to-end neural network on the first averaged PPG feature set, generating a second averaged PPG feature set of a second plurality of averaged PPG feature sets by applying a second average pooling layer of the end-to-end neural network on the second filtered PPG feature set, generating a third filtered PPG feature set of a third plurality of filtered PPG feature sets by applying a third convolutional layer of the end-to-end neural network on the second averaged PPG feature set, generating a PPG input of a PPG input sequence by applying a fourth convolutional layer of the end-to-end neural network on the third filtered PPG feature set, extracting a first LSTM feature set from the PPG input sequence by applying a first LSTM layer of the end-to-end neural network on the PPG input sequence, generating a second LSTM feature set by applying a second LSTM layer of the end-to-end neural network on the first LSTM feature set, generating a PPG fully connected feature set by applying a first fully connected layer of the end-to-end neural network on the second LSTM feature set, and obtaining estimated values of the systolic blood pressure and the diastolic blood pressure by applying a regression method on the PPG fully connected feature set through feeding the PPG fully connected feature set to a regression layer of the end-to-end neural network. An exemplary first convolutional layer may include a first plurality of convolution filters. In an exemplary embodiment, the second convolutional layer may include a second plurality of convolution filters. An exemplary third convolutional layer may include a third plurality of convolution filters and an exemplary fourth convolutional layer may include a fourth plurality of convolution filters. In an exemplary embodiment, the first LSTM layer may include a first plurality of LSTM units and the second LSTM layer may include a second plurality of LSTM units.

In an exemplary embodiment, applying the end-to-end neural network may further include providing a training data set, acquiring calibration values of the systolic blood pressure and the diastolic blood pressure of the subject, acquiring a standard ECG signal of the subject, providing an updated training data set by adding the calibration values and the standard ECG signal to the training data set, and training the end-to-end neural network utilizing the updated training data set. An exemplary training data set may be associated with the plurality of physiological parameters and may include the plurality of reference ECG signals and the plurality of reference PPG signals. In an exemplary embodiment, a cuff-based measurement method may be utilized to acquire the calibration values. In an exemplary embodiment, a plurality of ECG electrodes may be utilized to acquire the standard ECG signal.

In an exemplary embodiment, estimating the systolic blood pressure and the diastolic blood pressure may further include removing an estimation offset of the systolic blood pressure and the diastolic blood pressure by subtracting each calibration value of the systolic blood pressure and the diastolic blood pressure from a respective estimated value of the systolic blood pressure and the diastolic blood pressure.

In an exemplary embodiment, measuring each of the plurality of physiological parameters may further include estimating a body temperature of the plurality of physiological parameters by measuring a radiation power of a thermal radiation from the subject's body utilizing a thermopile sensor of the sensor package.

In an exemplary embodiment, measuring each of the plurality of physiological parameters may further include detecting a cough occurrence of the plurality of physiological parameters by recording an audio signal simultaneously with acquiring the motion signal and detecting the cough occurrence responsive to a magnitude of a motion sample of the motion signal being larger than a predetermined cough threshold, a center frequency of the audio signal being located in a predetermined frequency range, and a peak amplitude of the audio signal being larger than a predetermined amplitude threshold. An exemplary microphone may be utilized for recording the audio signal. In an exemplary embodiment, the audio signal may be associated with the motion sample of the motion signal.

In an exemplary embodiment, detecting the cough occurrence may include segmenting the audio signal to a plurality of audio segments by extracting each of the plurality of audio segments from the audio signal at a predefined time interval, segmenting the motion signal to a plurality of motion segments by extracting each of the plurality of motion segments from the motion signal at the predefined time interval, extracting a first filtered cough feature set of a first plurality of filtered cough feature sets from a first audio segment of the plurality of audio segments and a first motion segment of the plurality of motion segments by applying a fifth convolutional layer on the first audio segment and the first motion segment, generating a first averaged cough feature set of a first plurality of averaged cough feature sets by applying a third average pooling layer on the first cough feature set, generating a second filtered cough feature set of a second plurality of filtered cough feature sets by applying a sixth convolutional layer on the first averaged cough feature set, generating a second averaged cough feature set of a second plurality of averaged cough feature sets by applying a fourth average pooling layer on the second filtered cough feature set, generating a cough input of a cough input sequence by applying a seventh convolutional layer on the second averaged cough feature set, extracting a third LSTM feature set from the cough input sequence by applying a third LSTM layer on the cough input sequence, generating a fourth LSTM feature set by applying a fourth LSTM layer on the third LSTM feature set, generating a cough fully connected feature set by applying a second fully connected layer on the fourth LSTM feature set, and classifying the first audio segment in one of a cough event class or a non-cough event class by applying a classification method on the cough fully connected feature set through feeding the cough fully connected feature set to a classification layer. An exemplary fifth convolutional layer may include a fifth plurality of convolution filters. In an exemplary embodiment, the sixth convolutional layer may include a sixth plurality of convolution filters and the seventh convolutional layer may include a seventh plurality of convolution filters. An exemplary third LSTM layer may include a third plurality of LSTM units and an exemplary fourth LSTM layer comprising a fourth plurality of LSTM units.

In an exemplary embodiment, measuring each of the plurality of physiological parameters once per a respective time period may further include adjusting each respective time period of the plurality of time periods based on a measured value of a respective physiological parameter of the plurality of physiological parameters.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for health monitoring of a subject, consistent with one or more exemplary embodiments of the present disclosure.

FIG. 1B shows a flowchart of measuring an Oxygen saturation level (SpO₂), consistent with one or more exemplary embodiments of the present disclosure.

FIG. 1C shows a flowchart of estimating a respiratory rate, consistent with one or more exemplary embodiments of the present disclosure.

FIG. 1D shows a flowchart of installing a sensor package at an optimal location, consistent with one or more exemplary embodiments of the present disclosure.

FIG. 1E shows a flowchart of calculating a plurality of quality factors, consistent with one or more exemplary embodiments of the present disclosure.

FIG. 1F shows a flowchart of estimating a systolic blood pressure and a diastolic blood pressure, consistent with one or more exemplary embodiments of the present disclosure.

FIG. 1G shows a flowchart of applying an end-to-end neural network on a plurality of photoplethysmography (PPG) segments, consistent with one or more exemplary embodiments of the present disclosure.

FIG. 1H shows a flowchart of preliminary steps for applying an end-to-end neural network on a plurality of PPG segments, consistent with one or more exemplary embodiments of the present disclosure.

FIG. 1I shows a flowchart of detecting a cough occurrence, consistent with one or more exemplary embodiments of the present disclosure.

FIG. 1J shows a flowchart of detecting a cough occurrence utilizing an end-to-end neural network, consistent with one or more exemplary embodiments of the present disclosure.

FIG. 1K shows a flowchart of applying an end-to-end neural network on a plurality of audio segments and a plurality of motion segments, consistent with one or more exemplary embodiments of the present disclosure.

FIG. 2A shows a schematic of a system for health monitoring of a subject, consistent with one or more exemplary embodiments of the present disclosure.

FIG. 2B shows a block diagram of a sensor package, consistent with one or more exemplary embodiments of the present disclosure.

FIG. 3 shows a schematic of sensor package accessories, consistent with one or more exemplary embodiments of the present disclosure.

FIG. 4 shows a block diagram of a long short-term memory (LSTM) neural network, consistent with one or more exemplary embodiments of the present disclosure.

FIG. 5 shows a block diagram of a respiratory rate estimator, consistent with one or more exemplary embodiments of the present disclosure.

FIG. 6A shows a block diagram of an end-to-end neural network for blood pressure estimation, consistent with one or more exemplary embodiments of the present disclosure.

FIG. 6B shows a block diagram of convolutional neural network (CNN) layers for processing a plurality of PPG segments, consistent with one or more exemplary embodiments of the present disclosure.

FIG. 6C shows a block diagram of LSTM layers for generating estimated values of systolic blood pressure and diastolic blood pressure, consistent with one or more exemplary embodiments of the present disclosure.

FIG. 7A shows a block diagram of an end-to-end neural network for cough detection, consistent with one or more exemplary embodiments of the present disclosure.

FIG. 7B shows a block diagram of CNN layers for processing a plurality of audio segments and a plurality of motion segments, consistent with one or more exemplary embodiments of the present disclosure.

FIG. 7C shows a block diagram of LSTM layers for detecting a cough occurrence, consistent with one or more exemplary embodiments of the present disclosure.

FIG. 8 shows a high-level functional block diagram of a computer system, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for healthcare monitoring. An exemplary system may include a sensor package that includes multiple sensors and a low cost processing unit for data acquisition and transmission. An exemplary processing unit may receive a number of biomedical signals from a subject, including electrocardiogram (ECG), photoplethysmogram (PPG), voice (i.e., audio signal), motion signal, and body temperature, through different sensors and may send them for further processing to a conventional processor. An exemplary conventional processor may include a commercial electronic device such as a mobile phone, a portable (tablet or laptop) computer, a personal computer, etc. An exemplary processor may process the acquired signals utilizing various machine-learning methods to monitor different physiological parameters of the subject, such as arrhythmia, heart rate, blood pressure, respiratory rate, and cough occurrences. Exemplary deep neural networks may be utilized for parameter estimation due to a cost-efficient implementation of such structures after being trained, which may facilitate implementing a real-time health monitoring system on a commercial processor. Besides, exemplary deep neural networks may be able to extract appropriate representations from various data types.

Based on estimated values of the physiological parameters, the frequency of signal measurements and estimations may be modified so that unnecessary measurements may be avoided. As a result, power consumption of the system may be optimized, which leads to reducing an overall cost of the system.

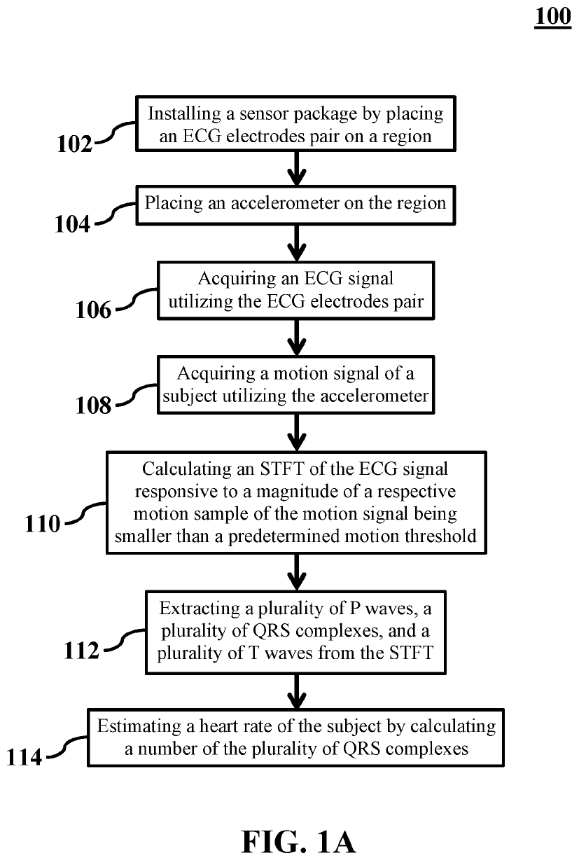

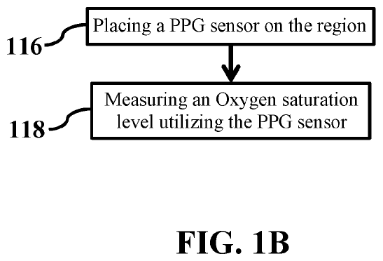

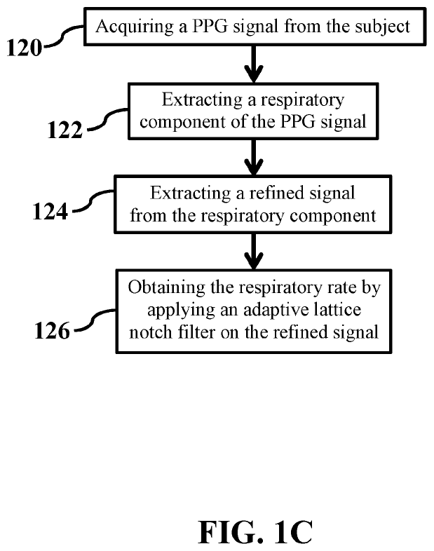

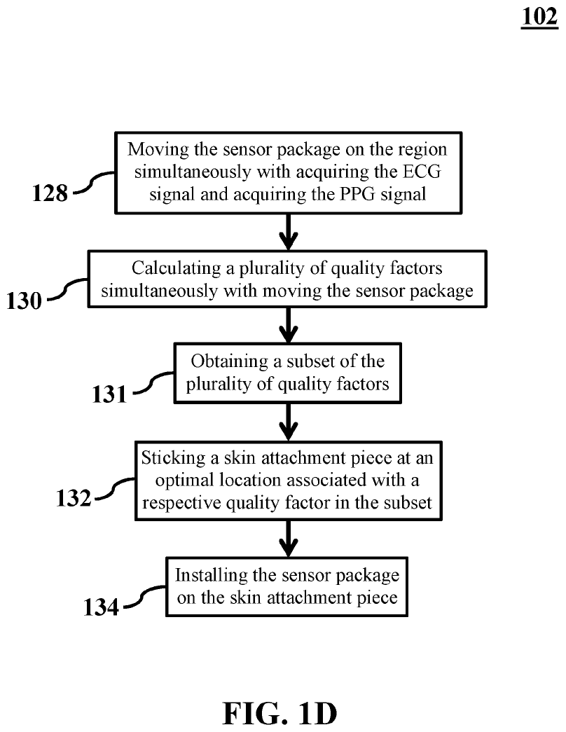

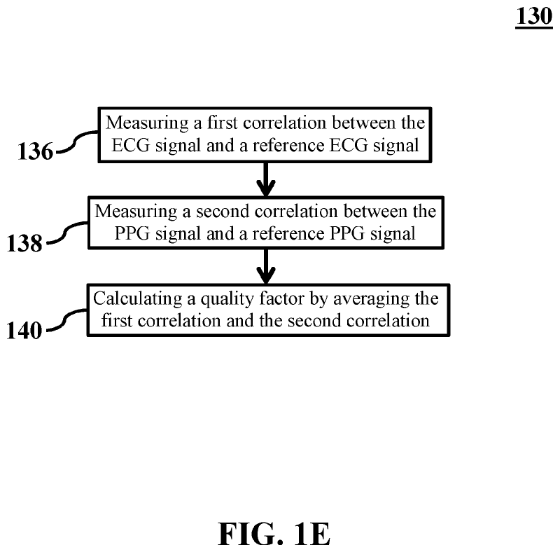

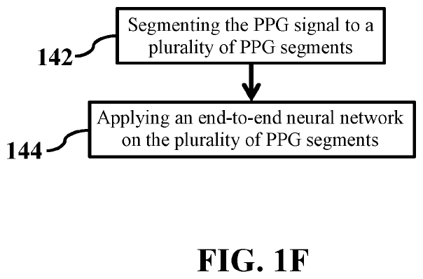

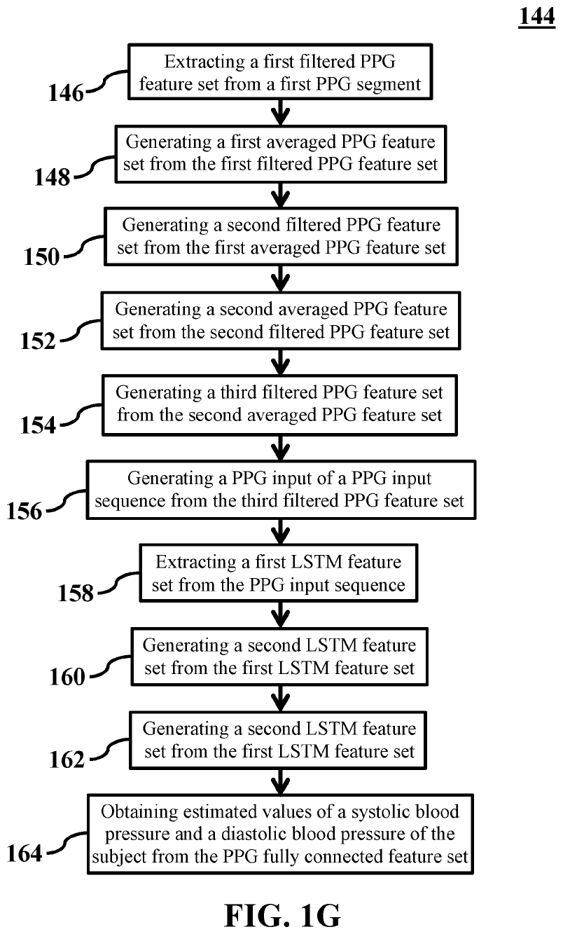

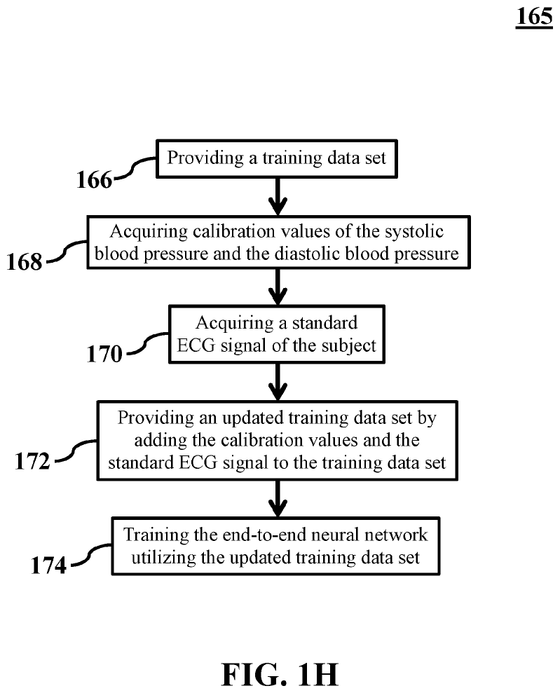

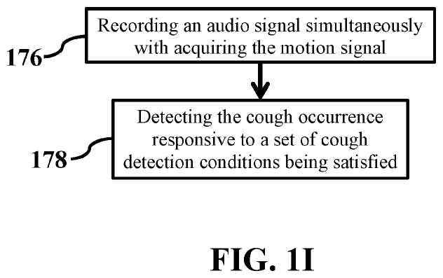

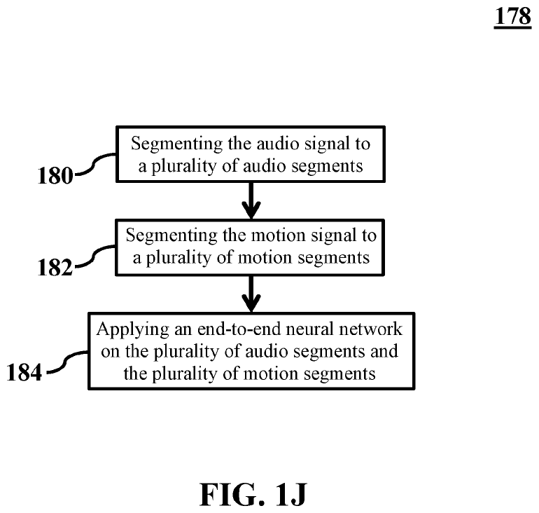

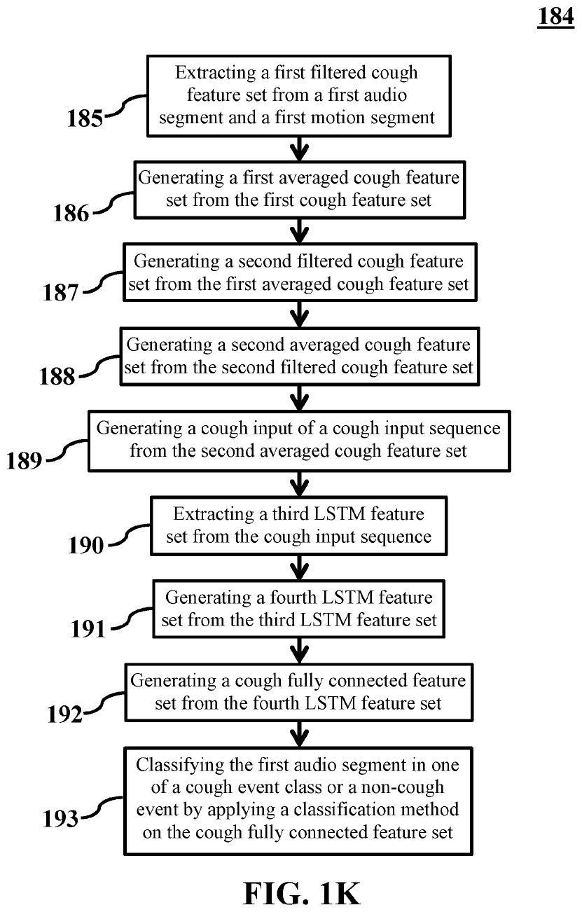

FIG. 1A shows a flowchart of a method for health monitoring of a subject, consistent with one or more exemplary embodiments of the present disclosure. An exemplary method 100 may include measuring each of a plurality of physiological parameters of the subject once per a respective time period of a plurality of time periods. In an exemplary embodiment, measuring each of the plurality of physiological parameters may include measuring a heart rate of the plurality of physiological parameters. In an exemplary embodiment, measuring the heart rate may include installing a sensor package by placing an electrocardiography (ECG) electrodes pair of the sensor package on a region at a right side of a chest of the subject (step 102), placing an accelerometer of the sensor package on the region (step 104), acquiring an ECG signal of the subject by acquiring each ECG sample of the ECG signal utilizing the ECG electrodes pair (step 106), acquiring a motion signal of the subject utilizing the accelerometer by acquiring each motion sample of the motion signal simultaneously with acquiring a respective ECG sample of the ECG signal (step 108), calculating a short-time Fourier transform (STFT) of the ECG signal responsive to a magnitude of a respective motion sample of the motion signal being smaller than a predetermined motion threshold (step 110), extracting a plurality of P waves, a plurality of QRS complexes, and a plurality of T waves from the STFT by applying a long short-term memory (LSTM) neural network on the STFT (step 112), and estimating the heart rate by calculating a number of the plurality of QRS complexes in a given period of time (step 114).

In an exemplary embodiment, measuring each of the plurality of physiological parameters in method 100 may further include measuring an Oxygen saturation level (SpO₂) of the plurality of physiological parameters. FIG. 1B shows a flowchart of measuring SpO₂, consistent with one or more exemplary embodiments of the present disclosure. In an exemplary embodiment, measuring the SpO₂ may include placing a photoplethysmography (PPG) sensor of the sensor package on the region (step 116) and measuring the SpO₂ utilizing the PPG sensor (step 118).

In an exemplary embodiment, measuring each of the plurality of physiological parameters in method 100 may further include estimating a respiratory rate of the plurality of physiological parameters. FIG. 1C shows a flowchart of estimating a respiratory rate, consistent with one or more exemplary embodiments of the present disclosure. In an exemplary embodiment, estimating the respiratory rate may include acquiring a PPG signal from the subject (step 120), extracting a respiratory component of the PPG signal responsive to a magnitude of a respective motion sample of the motion signal being smaller than the predetermined motion threshold (step 122), extracting a refined signal from the respiratory component by applying a band-pass filter on the respiratory component (step 124), and obtaining the respiratory rate by applying an adaptive lattice notch filter (ALNF) on the refined signal (step 126).

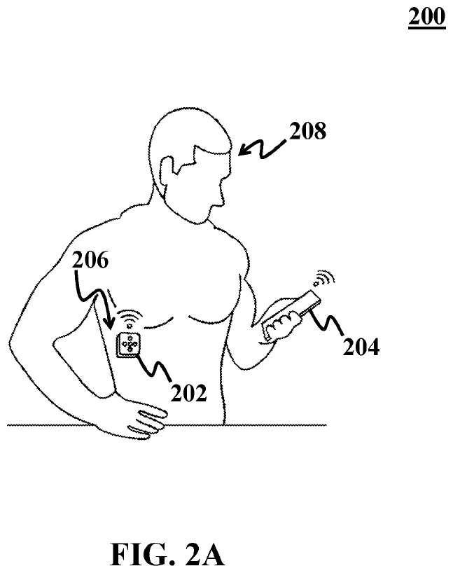

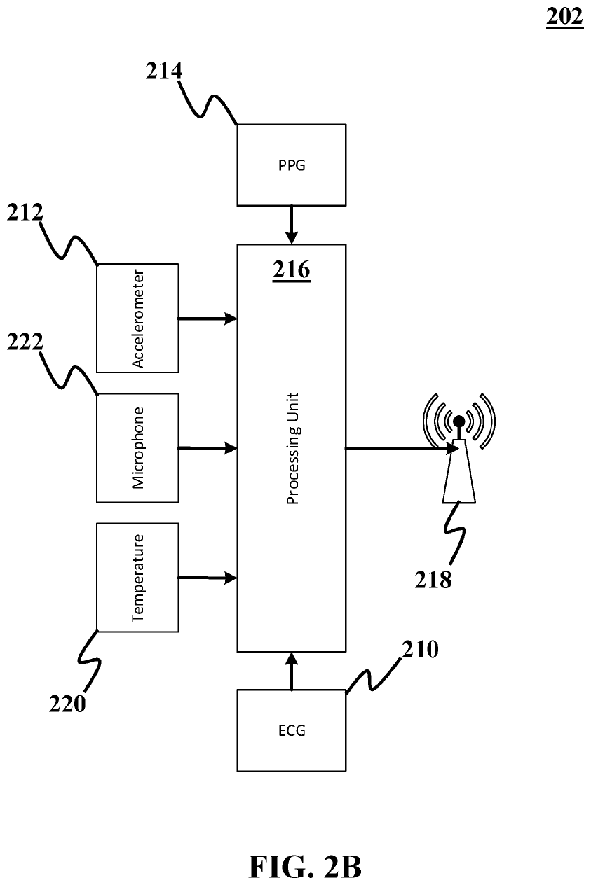

FIG. 2A shows a schematic of a system for health monitoring of a subject, consistent with one or more exemplary embodiments of the present disclosure. In an exemplary embodiment, different steps of method 100 may be implemented utilizing an exemplary system 200. In an exemplary embodiment, system 200 may include a sensor package 202 and a processor 204. In an exemplary embodiment, sensor package 202 may be installed at a region 206 at a right side of a chest of a subject 208.

In further detail with respect to sensor package 202, FIG. 2B shows a block diagram of a sensor package, consistent with one or more exemplary embodiments of the present disclosure. In an exemplary embodiment, sensor package 202 may include an ECG electrodes pair 210, an accelerometer 212, and a PPG sensor 214. In an exemplary embodiment, sensor package 202 may further include a processing unit 216 that may capture and send acquired physiological signals via a transmission unit 218 to processor 204 for further processing of the signals that may be required for estimating different physiological parameters. Therefore, in an exemplary embodiment, a cost-efficient processor may be utilized to implement processing unit 216 which may be adequate for signal acquisition and transmission, reducing an overall cost of sensor package 202. In an exemplary embodiment, transmission unit 218 may include a telecommunication device, such as a Bluetooth or wireless device, to send data from processing unit 216 to processor 204. In an exemplary embodiment, different commercial electronic devices may be utilized to implement processor 204, such as smartphones, tablet computers, PCs, etc.

In an exemplary embodiment, installing sensor package 202 in step 102 may include placing ECG electrodes pair 210 on region 206. In an exemplary embodiment, ECG electrodes pair 210 may include a pair of biocompatible cohesive ECG electrodes that may be placed about one inch apart on region 206 in a vertical orientation. An exemplary vertical orientation of ECG electrodes pair 210 may improve the quality of acquired ECG signals. In an exemplary embodiment, region 206 may include a right serratus anterior muscle of subject 208. Since, in an exemplary embodiment, a single-lead ECG may be acquired by utilizing two ECG electrodes pair 210 that may be placed only about one-inch apart, the quality of acquired ECG signals may be lower than signals obtained from standard ECG leads. Therefore, in an exemplary embodiment, region 206 may be preferred over other parts of the subject's body because ECG signals acquired from this area may convey more information than signals acquired from other areas. In addition, in an exemplary embodiment, a core body temperature may be measured more precisely at region 206 than other areas, particularly due to a reduced impact of movement artifacts on recorded physiological signals. In addition, exemplary movements detected in region 206 may have a higher correlation with physiological parameters of subject 208, such as coughing. Therefore, such movements may be utilized for better estimation of physiological parameters. Furthermore, in an exemplary embodiment, skin movement and elongation may be limited in region 206, which may facilitate providing a comfortable and durable wearable device.

In an exemplary embodiment, accurate placement of sensor package 202 may be vital to have precise data acquisition. Therefore, an exemplary placement mechanism may help intended users including healthcare professionals and laypersons. In an exemplary embodiment, balancing between weight bearing adhesive to hold sensor package 202 while avoiding excessive skin irritation and discomfort to subject 208 may be considered when designing a skin friendly sensor package.

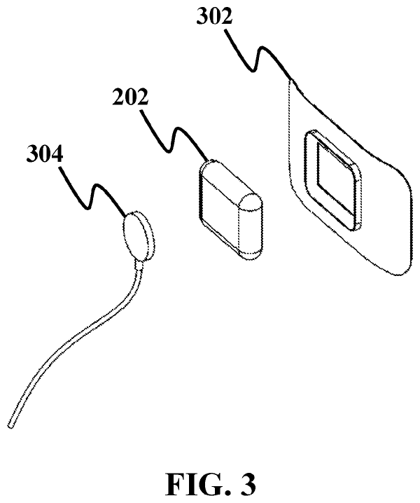

FIG. 3 shows a schematic of sensor package accessories, consistent with one or more exemplary embodiments of the present disclosure. Exemplary sensor package accessories may include a skin attachment piece 302 and an inductive or conductive charger 304. In an exemplary embodiment, skin attachment piece 302 may be attached to the subject's skin and sensor package 202 may be installed on region 206 through skin attachment piece 302. As a result, in an exemplary embodiment, sensor package 202 may be detached from skin attachment piece 302 and attached to inductive or conductive charger 304 to be recharged. Since skin attachment piece 302 may remain attached to the subject's skin when sensor package 202 is detached, there may be no need to relocate an attachment point in region 206 for installing sensor package 202. Therefore, in an exemplary embodiment, an optimal location may be found in the beginning of the installation process, so that sensor package 202 may be reinstalled at the same location through attachment piece 302 without a need to relocate the attachment point, which may reduce a stress to the subject's skin while sensor package 202 is reinstalled. However, in an exemplary embodiment, sensor package 202 may be capable of working in a relatively wide area to avoid skin damage from long-term irritation of a single location.

For further detail with regards to step 102, FIG. 1D shows a flowchart of installing a sensor package at an optimal location, consistent with one or more exemplary embodiments of the present disclosure. In an exemplary embodiment, installing sensor package 202 in step 102 may further include moving sensor package 202 on region 206 simultaneously with acquiring the ECG signal and acquiring the PPG signal (step 128), calculating a plurality of quality factors simultaneously with moving sensor package 202 (step 130), obtaining a subset of the plurality of quality factors (step 131), sticking skin attachment piece 302 at an optimal location of a plurality of locations in region 206 (step 132), and installing sensor package 202 on skin attachment piece 302 (step 134). In an exemplary embodiment, each of the plurality of quality factors may be associated with a respective location of the plurality of locations.

In further detail regarding step 128, in an exemplary embodiment, system 200 may have an installation mode in which sensor package 202 may be moved by a user on region 206 (and particularly around the serratus anterior muscle of subject 208) to find and mark a right spot (i.e., an optimal location). Referring again to FIGS. 2A and 2B, in an exemplary embodiment, ECG electrodes pair 210 and PPG sensor 214 may remain in contact with the skin of subject 208 to continuously acquire ECG and PPG signals. These signals may be utilized to find an exemplary optimal location in region 206 for sensor package 202 installation.

For further detail with respect to step 130, FIG. 1E shows a flowchart of calculating a plurality of quality factors, consistent with one or more exemplary embodiments of the present disclosure. In an exemplary embodiment, calculating the plurality of quality factors in step 130 may include measuring a first correlation between the ECG signal and a reference ECG signal of a plurality of reference ECG signals (step 136), measuring a second correlation between the PPG signal and a reference PPG signal of a plurality of reference PPG signals (step 138), and calculating a quality factor of the plurality of quality factors by averaging the first correlation and the second correlation (step 140).

In further detail regarding steps 136 and 138, in an exemplary embodiment, the plurality of reference ECG signals and the plurality of reference PPG signals may be obtained from a database that may include standard ECG and PPG signals. Referring again to FIG. 2A, Exemplary reference ECG signals and reference PPG signals may be stored in a memory unit of processor 204 prior to initializing system 200. In an exemplary embodiment, the first correlation may be measured in real-time between a given segment of the ECG signal and a corresponding segment of the reference ECG signal (which may be selected from the plurality of reference ECG signals) while sensor package 202 is being moved on region 206. Similarly, in an exemplary embodiment, the second correlation may be measured in real-time between a given segment of the PPG signal and a corresponding segment of the reference PPG signal (which may be selected from the plurality of reference PPG signals). In an exemplary embodiment, different correlation coefficients may be calculated for the corresponding segments to measure the first correlation or the second correlation, such as Pearson correlation coefficient, rank correlation coefficients, etc.

In an exemplary embodiment, an average value of the first correlation and the second correlation may be calculated in step 140 as a quality factor to measure an overall quality of physiological signals that may be acquired at a specific point in region 206. In an exemplary embodiment, each of the plurality of quality factors may be obtained by performing steps 136, 138, and 140 for a different point (i.e., location) in region 206. As a result, in an exemplary embodiment, several points in region 206 may be assessed in terms of the quality of acquired physiological signals by assigning a separate quality factor to each point.

Referring again to FIGS. 1D, step 131 may include obtaining a subset of the plurality of quality factors. Each respective quality factor in an exemplary subset may include a value larger than a predetermined ratio of a largest quality factor of the plurality of quality factors. In an exemplary embodiment, obtaining the subset may include selecting each quality factor of the plurality of quality factors that may have a value larger than the predetermined ratio of the largest quality factor. An exemplary predetermined ratio may be 0.7 of the value of the largest quality factor. In an exemplary embodiment, the exact ratio for each quality factor for different individuals may be determined by system 200, based on a minimum quality required to extract an accurate output.

Referring again to FIGS. 1D and 3, step 132 may include sticking skin attachment piece 302 at the optimal location. An exemplary optimal location may be associated with a respective quality factor in the subset. In an exemplary embodiment, since the magnitude of each quality factor is proportional to the quality of physiological signals that may be acquired from the corresponding location, a quality factor which has a value larger than the predetermined ratio of the largest quality factor may be utilized to locate a point in region 206 that may be more suitable for installing sensor package 202 among the assessed points in region 206. Therefore, in an exemplary embodiment, skin attachment piece 302 may be stuck to the optimal location that may correspond to a selected quality factor in the subset. In an exemplary embodiment, different points in region 206 corresponding to different quality factors in the subset may be periodically selected as attachment locations to avoid skin damage from long-term irritation of a single location at which sensor package 202 may be installed.

In further detail regarding step 134, in an exemplary embodiment, sensor package 202 may be attached to skin attachment piece 302 after sticking skin attachment piece 302 to the optimal location. In an exemplary embodiment, this mechanism may eliminate a need for relocating sensor package 202 multiple times which may cause skin irritation, stripping and tension blisters. Moreover, it may provide a secure and versatile station for sensor package 202 that may enable a user to remove sensor package 202 for charging or required servicing and install it back at a same location without a need to repeat the relocation process, which may save time and reduce the computational cost of method 100.

Referring again to FIGS. 1A and 2B, step 104 may include placing accelerometer 212 on region 206. Since accelerometer 212 may be included inside sensor package 202, accelerometer 212 may also be installed when sensor package 202 is installed on the optimal location through skin attachment piece 302. However, in an exemplary embodiment, a position of accelerometer 212 may be adjusted after installing sensor package 202.

For further detail with respect to step 106, in an exemplary embodiment, ECG electrodes pair 210 may be utilized to acquire the ECG signal of subject 208. An exemplary ECG signal may include periodic waveforms and each of the periodic waveforms may be composed of different segments, including a P wave, a QRS complex, and a T wave. The shape of these waves may include vital information to analyze the heart conditions and detect different heart problems, such as an arrhythmia, enlargement of heart chambers, and a clogging up of blood vessels that may cause serious health issues.

In further detail with regards to step 108, in an exemplary embodiment, accelerometer 212 may be utilized to acquire the motion signal of subject 208. An exemplary motion signal may include a three dimensional signal which represents motions of subject 208 in different physical dimensions.

In further detail regarding step 110, in an exemplary embodiment, each motion sample of the motion signal may be compared with the predetermined motion threshold. An exemplary predetermined motion threshold may be empirically determined to distinguish body movements due to biological causes (such as respiration, cough, etc.) from motion artifacts. In an exemplary embodiment, if the magnitude of the motion sample is lower than the predetermined motion threshold, method 100 may proceed to calculate the STFT of the ECG signal. In an exemplary embodiment, the STFT may be calculated by applying an STFT transform on the ECG signal. In an exemplary embodiment, useful time-frequency features may be extracted from the ECG signal by applying the STFT transform, which may be utilized for detecting different segments of the ECG signal. In an exemplary embodiment, segmentation of different regions of the ECG signal may provide a basis for measurements useful for assessing an overall health and well-being of human heart and detecting abnormalities.

In an exemplary embodiment, if the magnitude of the motion sample is higher than the predetermined motion threshold, method 100 may detect a serious motion artifact and not proceed further until the artifact is removed. An exemplary artifact removal may be determined by a reduction of the magnitude of the motion signal to a value lower than the predetermined motion threshold.

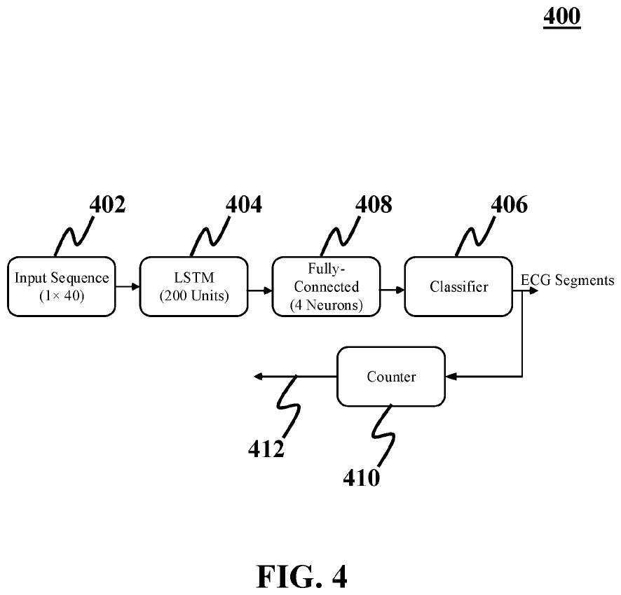

For further detail with respect to step 112, FIG. 4 shows a block diagram of an LSTM neural network, consistent with one or more exemplary embodiments of the present disclosure. An exemplary LSTM neural network 400 may be capable of tracking long-term dependencies in an input sequence 402. In an exemplary embodiment, a single input of input sequence 402 may include a windowed segment of the STFT. An exemplary Kaiser window of length 128 may be applied on the STFT to extract the windowed segment. In an exemplary embodiment, the windowed segment may be fed to an LSTM layer 404 that includes LSTM units. An exemplary output of LSTM layer 404 may be applied to a classifier 406 through a fully-connected layer 408 composed of neurons. In an exemplary embodiment, classifier 406 may classify the windowed segment of the ECG signal based on an output data of fully-connected layer 408 in four classes of P (corresponding to a P wave), T (corresponding to a T wave), QRS (corresponding to a QRS complex), and n/a to label samples that do not belong to any region of interest in the ECG signal. Therefore, in an exemplary embodiment, a P wave, a QRS complex, or a T wave may be extracted from each windowed segment of the STFT. As a result, different segments of the ECG signal may be detected and labeled by extracting the plurality of P waves, the plurality of QRS complexes, and the plurality of T waves from the STFT. This information may help a physician to understand the heart condition of subject 208 and detect potential abnormalities. Exemplary numerical values for different layers of LSTM neural network 400 for a sampling rate of 125 Hz are shown in FIG. 4.

In further detail with regards to step 114, the heart rate may be estimated after identifying the plurality of QRS complexes. An exemplary counter unit 410 may be configured to count a number of successive occurrences of the plurality of QRS complexes during a given period of time (for example, one minute), which may be determined as an estimated heart rate 412.

Referring again to FIGS. 1B and 2B, step 116 may include placing PPG sensor 214 on region 206. Since PPG sensor 214 may be included inside sensor package 202, PPG sensor 214 may also be installed when sensor package 202 is installed on the optimal location through skin attachment piece 302. However, in an exemplary embodiment, a position of PPG sensor 214 may be adjusted after installing sensor package 202.

In further detail with regarding step 118, in an exemplary embodiment, PPG sensor 214 may be configured to measure the SpO₂ by shining red and then near-infrared wavelengths through vascular tissues of subject 208 with fast switching between the red and the near-infrared wavelengths. Amplitudes of the red and near-infrared AC signals may be sensitive to SpO₂ variations due to differences in light absorption of oxyhaemoglobin (HbO₂) and reduced haemoglobin (Hb) at red and near-infrared wavelengths. In an exemplary embodiment, the SpO₂ may be estimated from the amplitude ratio of the red AC signals to the near-infrared AC signals and corresponding DC components of the PPG signal.

Referring again to FIGS. 1C, 2A, and 2B, in an exemplary embodiment, step 120 may include acquiring the PPG signal from subject 208. In an exemplary embodiment, acquiring the PPG signal may include acquiring each PPG sample of the PPG signal utilizing PPG sensor 214 simultaneously with acquiring a respective ECG sample of the ECG signal.

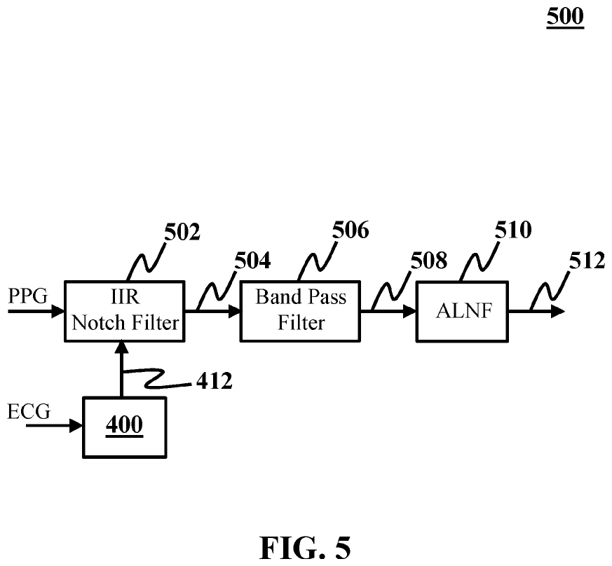

For further detail with respect to step 122-126, FIG. 5 shows a block diagram of a respiratory rate estimator, consistent with one or more exemplary embodiments of the present disclosure. An exemplary respiratory rate estimator 500 may be utilized to extract respiratory rate from PPG signals based on the assumption that respiration may modulate a PPG baseline. Therefore, in an exemplary embodiment, respiratory rate may be estimated from PPG using a frequency estimator based on an adaptive notch filter. A band-pass frequency of an exemplary notch filter may be adjusted based on the heart rate. An exemplary adaptive notch filter may estimate dominant frequencies related to cardiac components in a PPG signal. Therefore, in an exemplary embodiment, the adaptive notch filter may be used to decompose the PPG signal into its two main dominant components, including cardiac and respiratory signals. After cardiac component removal, a band-pass filter may be utilized to refine the residual signal. Afterwards, an exemplary respiratory rate may be stably estimated by employing a frequency estimator.

For further detail with regards to 126, in an exemplary embodiment, respiratory rate estimator 500 may include a sequential harmonic infinite impulse response (IIR) notch filter 502 that may be configured to remove a cardiac component of the PPG signal based on estimated heart rate 412. In an exemplary embodiment, IIR notch filter 502 may receive estimated heart rate 412 from LSTM neural network 400 and generate a residual 504 of the PPG signal by removing the cardiac component. Since, in an exemplary embodiment, estimated heart rate 412 may be obtained from the ECG signal, results may be more accurate than conventional methods that utilize PPG signals for heart rate estimation. Furthermore, in an exemplary embodiment, utilizing estimated heart rate 412 may reduce computational costs since there may be no further need to recalculate the heart rate which is already estimated in previous steps of method 100.

In further detail regarding step 124, in an exemplary embodiment, respiratory rate estimator 500 may further include a band-pass filter 506 that may generate a refined signal 508 by filtering residual 504 at a given frequency range. In an exemplary embodiment, band-pass filter 506 may be configured to pass frequencies in a range of about (0.1, 2) Hz.

For further detail with regards to step 126, in an exemplary embodiment, respiratory rate estimator 500 may further include an ALNF 510. In an exemplary embodiment, ALNF 510 may refer to an adaptive IIR notch filter that may be combined with a lattice form which may be utilized for frequency estimation. In an exemplary embodiment, ALNF 510 may be configured to obtain an estimated a respiratory rate 512 from refined signal 508.

In an exemplary embodiment, measuring each of the plurality of physiological parameters in method 100 may further include estimating a systolic blood pressure of the plurality of physiological parameters and a diastolic blood pressure of the plurality of physiological parameters in each cardiac cycle. FIG. 1F shows a flowchart of estimating a systolic blood pressure and a diastolic blood pressure, consistent with one or more exemplary embodiments of the present disclosure. In an exemplary embodiment, estimating the systolic blood pressure and the diastolic blood pressure may include segmenting the PPG signal to a plurality of PPG segments (step 142) and applying an end-to-end neural network on the plurality of PPG segments (step 144).

In further detail with respect to step 142, in an exemplary embodiment, segmenting the PPG signal to the plurality of PPG segments may include extracting each of the plurality of PPG segments from the PPG signal at a respective time interval. An exemplary time interval may correspond to a respective QRS complex of the plurality of QRS complexes. Therefore, in an exemplary embodiment, each PPG segment may occur simultaneously with a corresponding QRS complex that may be utilized as a time reference. Each exemplary PPG segment may include two cycles and successive segments may overlap in one cycle. In an exemplary embodiment, zeros may be added to the end of each PPG segment to adjust the segment length to a predefined length.

For further detail regarding step 144, FIG. 1G shows a flowchart of applying an end-to-end neural network on a plurality of PPG segments, consistent with one or more exemplary embodiments of the present disclosure. In an exemplary embodiment, applying the end-to-end neural network on the plurality of PPG segments may include extracting a first filtered PPG feature set of a first plurality of filtered PPG feature sets from a PPG segment of the plurality of PPG segments (step 146), generating a first averaged PPG feature set of a first plurality of averaged PPG feature sets from the first filtered PPG feature set (step 148), generating a second filtered PPG feature set of a second plurality of filtered PPG feature sets from the first averaged PPG feature set (step 150), generating a second averaged PPG feature set of a second plurality of averaged PPG feature sets from the second filtered PPG feature set (step 152), generating a third filtered PPG feature set of a third plurality of filtered PPG feature sets from the second averaged PPG feature set (step 154), generating a PPG input of a PPG input sequence from the third filtered PPG feature set (step 156), extracting a first LSTM feature set from the PPG input sequence (step 158), generating a second LSTM feature set from the first LSTM feature set (step 160), generating a PPG fully connected feature set from the second LSTM feature set (step 162), and obtaining estimated values of the systolic blood pressure and the diastolic blood pressure from the PPG fully connected feature set (step 164).

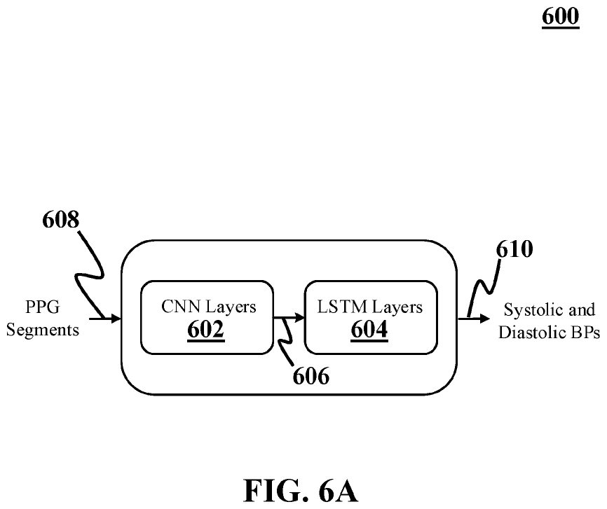

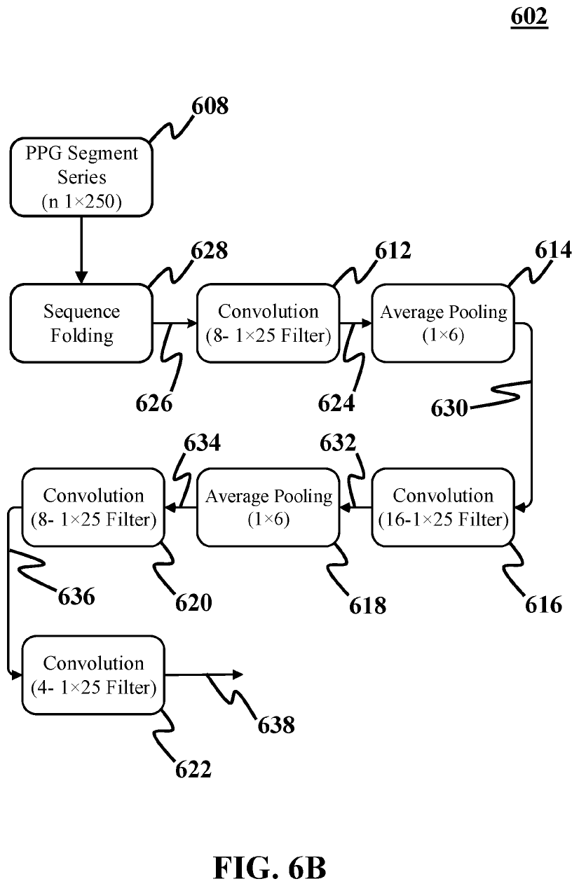

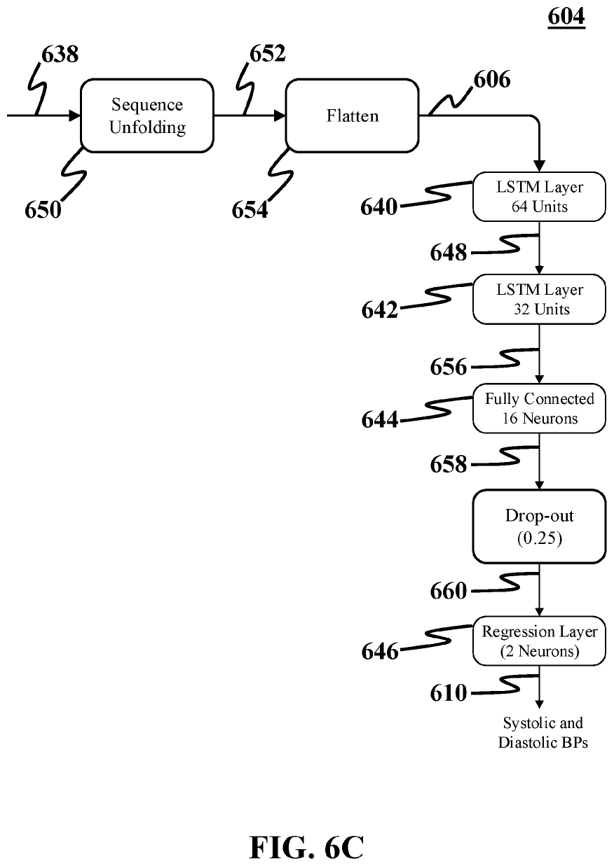

FIG. 6A shows a block diagram of an end-to-end neural network for blood pressure estimation, consistent with one or more exemplary embodiments of the present disclosure. In an exemplary embodiment, different steps of flowchart 144 of FIG. 1G may be implemented utilizing an exemplary end-to-end neural network 600. In an exemplary embodiment, end-to-end neural network 600 may include two hierarchy levels. An exemplary lower hierarchy level may include convolutional neural network (CNN) layers 602 and an exemplary upper hierarchy level may employ a two-stage LSTM network to account for time-domain variations of machine-learned features extracted by the lower hierarchy. An exemplary two-stage LSTM network may include LSTM layers 604. In an exemplary embodiment, CNN layers 602 may be configured to generate a PPG input sequence 606 that may include morphological features from a plurality of PPG segments 608. In an exemplary embodiment, PPG input sequence 606 may be fed to LSTM layers 604 to generate estimated values 610 of the systolic blood pressure and the diastolic blood pressure.

FIG. 6B shows a block diagram of CNN layers for processing a plurality of PPG segments, consistent with one or more exemplary embodiments of the present disclosure. In an exemplary embodiment, CNN layers 602 may include a first convolutional layer 612, a first average pooling layer 614, a second convolutional layer 616, a second average pooling layer 618, a third convolutional layer 620, and a fourth convolutional layer 622. In an exemplary embodiment, steps 146-156 may be implemented utilizing CNN layers 602. Exemplary numerical values for different layers of CNN layers 602 for a sampling rate of 125 Hz are shown in FIG. 6B.

For further detail with respect to step 146, an exemplary first filtered PPG feature set 624 may be extracted from a PPG segment 626 by applying first convolutional layer 612 on PPG segment 626. An exemplary sequence folding layer 628 may be applied on plurality of PPG segments 608 to extract PPG segment 626 from plurality of PPG segments 608 by splitting plurality of PPG segments 608. In an exemplary embodiment, first convolutional layer 612 may include a first plurality of convolution filters that may be configured to perform convolution operations on their respective inputs.

In further detail with regards to step 148, an exemplary first averaged PPG feature set 630 may be generated by applying first average pooling layer 614 on first filtered PPG feature set 624. In an exemplary embodiment, first average pooling layer 614 may be configured to reduce the size of first filtered PPG feature set 624 by replacing different subsets of first filtered PPG feature set 624 with average values of elements in the subsets.

For further detail regarding step 150, an exemplary second filtered PPG feature set 632 may be generated by applying second convolutional layer 616 on first averaged PPG feature set 630. In an exemplary embodiment, second convolutional layer 616 may include a second plurality of convolution filters that may be configured to perform convolution operations on their respective inputs.

In further detail with respect to step 152, an exemplary second averaged PPG feature set 634 may be generated by applying second average pooling layer 618 on second filtered PPG feature set 632. In an exemplary embodiment, second average pooling layer 618 may be configured to reduce the size of second filtered PPG feature set 632 by replacing different subsets of second filtered PPG feature set 632 with average values of elements in the subsets.

For further detail with regards to step 154, an exemplary third filtered PPG feature set 636 may be generated by applying third convolutional layer 620 on second averaged PPG feature set 634. In an exemplary embodiment, third convolutional layer 620 may include a third plurality of convolution filters that may be configured to perform convolution operations on their respective inputs.

In further detail regarding step 156, an exemplary PPG input 638 of PPG input sequence 606 may be generated by applying fourth convolutional layer 622 on third filtered PPG feature set 636. In an exemplary embodiment, fourth convolutional layer 622 may include a fourth plurality of convolution filters that may be configured to perform convolution operations on their respective inputs. As a result, in an exemplary embodiment, PPG input 638 may be obtained from PPG segment 626 by applying CNN layers 602 on PPG segment 626. Other exemplary PPG inputs of PPG input sequence 606 may be similarly obtained.

FIG. 6C shows a block diagram of LSTM layers for generating estimated values of systolic blood pressure and diastolic blood pressure, consistent with one or more exemplary embodiments of the present disclosure. In an exemplary embodiment, LSTM layers 604 may include a first LSTM layer 640, a second LSTM layer 642, a first fully connected layer 644, and a regression layer 646. In an exemplary embodiment, steps 158-164 may be implemented utilizing LSTM layers 604. Exemplary numerical values for different layers of LSTM layers 604 for a sampling rate of 125 Hz are shown in FIG. 6C.

For further detail with respect to step 158, an exemplary first LSTM feature set 648 may be extracted from PPG input sequence 606 by applying first LSTM layer 640 on PPG input sequence 606. In an exemplary embodiment, PPG input sequence 606 may be obtained by merging receiving each of PPG inputs (for example, PPG input 638) and merging the received PPG inputs. An exemplary sequence unfolding layer 650 may be applied on each PPG input to generate merged PPG inputs 652. An exemplary flatten layer 654 may receive and convert merged PPG inputs 652 to PPG input sequence 606. In an exemplary embodiment, first LSTM layer 640 may include a first plurality of LSTM units.

In further detail with regards to step 160, an exemplary second LSTM feature set 656 may be generated from first LSTM feature set 648 by applying second LSTM layer 642 on first LSTM feature set 648. In an exemplary embodiment, second LSTM layer 642 may include a second plurality of LSTM units.

For further detail regarding step 162, an exemplary PPG fully connected feature set 658 may be generated from second LSTM feature set 656 by applying first fully connected layer 644 on second LSTM feature set 656. In an exemplary embodiment, first fully connected layer 644 may include a number of neurons that may be configured to receive and process respective data from every neuron in second LSTM layer 642.

In further detail with respect to step 164, estimated values 610 of the systolic blood pressure and the diastolic blood pressure may be obtained by applying an exemplary regression method on PPG fully connected feature set 658. In an exemplary embodiment, regression layer 646 may include a number of neurons that may be configured to apply the regression method on PPG fully connected feature set 658.

In an exemplary embodiment, some kinds of personal calibration may be required to increase the estimation precision of physiological parameters for each specific individual (for example, subject 208). For instance, since a cardiovascular dynamic of each subject may be unique, the relationship between PPG signals and blood pressure may also be specific to each individual. Therefore, applying an effective strategy for calibration may increase accuracy of blood pressure estimation. Moreover, ECG segmentation results may also be more reliable if ECG signals of each subject (for example, subject 208) is included in a training data set that may be utilized for training end-to-end neural network 600 since the QRS segments of the ECG signal may affect the accuracy of PPG signal segmentation, as discussed in step 142 above. As a result, in an exemplary embodiment, preliminary steps may be performed for a personal calibration end-to-end neural network 600.

For further detail regarding step 144, FIG. 111 shows a flowchart of preliminary steps for applying an end-to-end neural network on a plurality of PPG segments, consistent with one or more exemplary embodiments of the present disclosure. Exemplary preliminary steps 165 may include providing a training data set (step 166), acquiring calibration values of the systolic blood pressure and the diastolic blood pressure of subject 208 (step 168), acquiring a standard ECG signal of subject 208 (step 170), providing an updated training data set by adding the calibration values and the standard ECG signal to the training data set (step 172), and training the end-to-end neural network utilizing the updated training data set (174).

For further detail regarding step 166, an exemplary training data set may be associated with the plurality of physiological parameters. In an exemplary embodiment, given values of physiological parameters and corresponding physiological signals, including the plurality of reference ECG signals and the plurality of reference PPG signals, may be provided from standard databases.

In further detail with respect to steps 168 and 170, an exemplary cuff-based measurement method may be utilized in step 168 to acquire calibration values of the systolic blood pressure and the diastolic blood pressure of subject 208. In an exemplary embodiment, the systolic blood pressure and the diastolic blood pressure of subject 208 may be measured continuously for a given time period (for example, about fifteen minutes). In an exemplary embodiment, a plurality of ECG electrodes may be utilized in step 170 to acquire the standard ECG signal. In an exemplary embodiment, conventional methods and devices may be utilized to obtain the calibration values and the standard ECG signal.

For further detail with regards to step 172, adding the calibration values and the standard ECG signal to the training data set may customize the database for subject 208 since the calibration values are specifically acquired from subject 208. As a result, an exemplary updated data set may be provided to calibrate end-to-end neural network 600 for subject 208.

In further detail regarding step 174, in an exemplary embodiment, end-to-end neural network 600 may be trained by feeding standard physiological signals (for example, the plurality of reference ECG signals and the plurality of reference PPG signals) in the updated training data set to end-to-end neural network 600 as training input data and using given values of physiological parameters that may correspond to the training input data as training output data. Referring again to FIG. 6C, an exemplary dropout layer 660 may be applied on output data of first fully connected layer 644 during the training process to prevent end-to-end neural network 600 from overfitting and improve an overall performance of the network.

For further detail regarding step 144, in an exemplary embodiment, estimating the systolic blood pressure and the diastolic blood pressure may further include removing an estimation offset of the systolic blood pressure and the diastolic blood pressure. An exemplary estimation offset may be removed by subtracting each calibration value of the systolic blood pressure and the diastolic blood pressure from a respective estimated value of estimated values 610. As a result, blood pressure estimation accuracy may be increased for a specific individual for example, subject 208) through a single-point calibration.

In an exemplary embodiment, measuring each of the plurality of physiological parameters in method 100 may further include estimating a body temperature of the plurality of physiological parameters. Referring again to FIG. 2B, in an exemplary embodiment, sensor package 202 may further include a thermopile sensor 220 that may be configured to measure a temperature of the body of subject 208 by measuring a radiation power of a thermal radiation from the subject's body.

In an exemplary embodiment, measuring each of the plurality of physiological parameters in method 100 may further include detecting a cough occurrence of the plurality of physiological parameters. Coughs are common symptoms of many respiratory diseases yet difficult to analyze. Automatic detection of cough sound may require filtering ambient noise and distinguishing them from other patient sounds, for instance, laughter, speech, and sneezing. Utilizing an exemplary accelerometer may be utilized for removing ambient noise and distinguishing coughs from other patient-related sounds more effectively, as well as detecting abrupt movements caused by coughing. Such information may be used to process voice signals. Similarly, an exemplary patient voice may help to distinguish chest movements caused by coughing from other movements.

FIG. 1I shows a flowchart of detecting a cough occurrence, consistent with one or more exemplary embodiments of the present disclosure. In an exemplary embodiment, detecting the cough occurrence may include recording an audio signal simultaneously with acquiring the motion signal (step 176) and detecting the cough occurrence responsive to a set of cough detection conditions being satisfied (step 178).

For further detail with regards to step 176, recording the audio signal may include utilizing an exemplary microphone. Referring again to FIG. 2B, in an exemplary embodiment, sensor package 202 may further include a microphone 222 that may be configured to record the audio signal. In an exemplary embodiment, the audio signal may be recorded simultaneously with the motion signal by activating microphone 222 while accelerometer 212 is recording the motion signal.

In further detail with respect to step 178, the cough occurrence may be detected if an exemplary set of cough detection conditions is satisfied. In an exemplary embodiment, the set may include a magnitude of a motion sample of the motion signal being larger than a predetermined cough threshold, a center frequency of the audio signal being located in a predetermined frequency range, and a peak amplitude of the audio signal being larger than a predetermined amplitude threshold. In an exemplary embodiment, the audio signal may be associated with the motion sample of the motion signal since different audio signals that are related to subject 208 may cause different motions. Therefore, an exemplary predetermined cough threshold may be set based on practical motion when a person coughs to effectively distinguish abrupt chest movements caused by coughing from other movements of subject 208. In an exemplary embodiment, the predetermined frequency range may be determined based on spectral features of cough to distinguish a cough occurrence from other sounds related to subject 208, for instance, laughter, speech, and sneezing. An exemplary predetermined amplitude threshold may be set based on the intensity of ordinary human cough to distinguish cough signals from other audio signals and remove ambient noise.

In an exemplary embodiment, an end-to-end neural network may be trained and utilized to adjust the above mentioned thresholds for cough detection conditions and implement step 178 for detecting a cough occurrence of subject 208. FIG. 1J shows a flowchart of detecting a cough occurrence utilizing an end-to-end neural network, consistent with one or more exemplary embodiments of the present disclosure. In an exemplary embodiment, detecting the cough occurrence in step 178 may include segmenting the audio signal to a plurality of audio segments (step 180), segmenting the motion signal to a plurality of motion segments (step 182), and applying an end-to-end neural network on the plurality of audio segments and the plurality of motion segments (step 184).

In further detail with respect to step 180, in an exemplary embodiment, segmenting the audio signal to the plurality of audio segments may include extracting each of the plurality of audio segments from the audio signal at a predefined time interval. An exemplary predefined time interval may be set based on an expected duration of a cough occurrence so that enough data is included in each audio segment for detecting a cough occurrence.

For further detail with regards to step 182, in an exemplary embodiment, segmenting the motion signal to the plurality of motion segments may include extracting each of the plurality of motion segments from the motion signal at the predefined time interval. When an exemplary motion signal includes three elements corresponding to the three physical dimensions, all three elements may be segmented. In an exemplary embodiment, the duration of each motion segment may be set equal to the duration of a corresponding audio segment since the motion of subject 208 may be correlated with the cough sound during when subject 208 coughs. Therefore, in an exemplary embodiment, each motion segment may occur simultaneously with a corresponding audio segment during a cough occurrence. An exemplary segment duration may be set to about 6 ms.

In further detail regarding step 184, FIG. 1K shows a flowchart of applying an end-to-end neural network on a plurality of audio segments and a plurality of motion segments, consistent with one or more exemplary embodiments of the present disclosure. In an exemplary embodiment, applying the end-to-end neural network on the plurality of audio segments and the plurality of motion segments may include extracting a first filtered cough feature set of a first plurality of filtered cough feature sets from a first audio segment of the plurality of audio segments and a first motion segment of the plurality of motion segments (step 185), generating a first averaged cough feature set of a first plurality of averaged cough feature sets from the first cough feature set (step 186), generating a second filtered cough feature set of a second plurality of filtered cough feature sets from the first averaged cough feature set (step 187), generating a second averaged cough feature set of a second plurality of averaged cough feature sets from the second filtered cough feature set (step 188), generating a cough input of a cough input sequence from the second averaged cough feature set (step 189), extracting a third LSTM feature set from the cough input sequence (step 190), generating a fourth LSTM feature set from the third LSTM feature set (step 191), generating a cough fully connected feature set from the fourth LSTM feature set (step 192), and classifying the first audio segment in one of a cough event class or a non-cough event class by applying a classification method on the cough fully connected feature set (step 193).

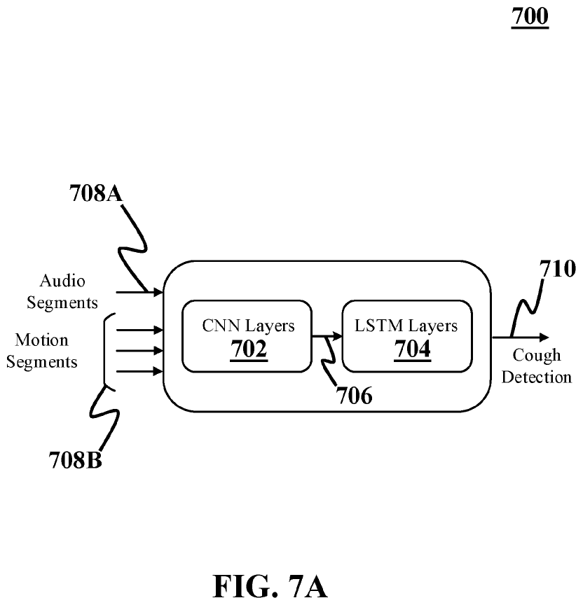

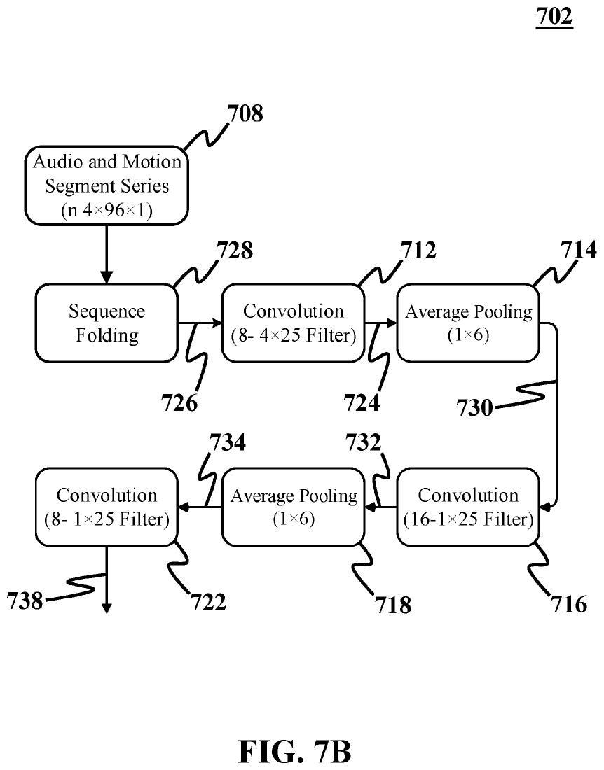

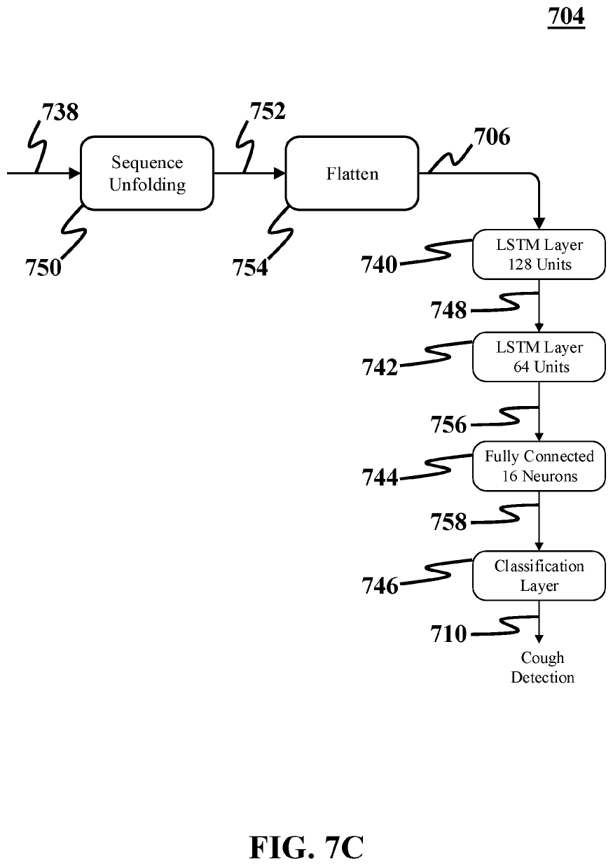

FIG. 7A shows a block diagram of an end-to-end neural network for cough detection, consistent with one or more exemplary embodiments of the present disclosure. In an exemplary embodiment, different steps of flowchart 184 of FIG. 1K may be implemented utilizing an exemplary end-to-end neural network 700. In an exemplary embodiment, end-to-end neural network 700 may include two hierarchy levels. An exemplary lower hierarchy level may include CNN layers 702 and an exemplary upper hierarchy level may employ a two-stage LSTM network to account for time-domain variations of machine-learned features extracted by the lower hierarchy. An exemplary two-stage LSTM network may include LSTM layers 704. In an exemplary embodiment, CNN layers 702 may be configured to generate a cough input sequence 706 from a plurality of audio segments 708A and a plurality of motion segments 708B. To adjust the size of each audio segment with a corresponding motion segment, in an exemplary embodiment, the motion signal may be upsampled since a sampling rate of the motion signal may be lower than that of the audio signal.

In an exemplary embodiment, cough input sequence 706 may include appropriate representations of signal segments. In an exemplary embodiment, cough input sequence 706 may be fed to LSTM layers 704 to generate a cough detection signal 710. In an exemplary embodiment, cough detection signal 710 may include two different values representing a cough event class and a non-cough event class. Therefore, in an exemplary embodiment, every audio segment of plurality of audio segments 708A may be classified to one of the cough event class and the non-cough event class by end-to-end neural network 700. As a result, in an exemplary embodiment, every cough occurrence of subject 208 may be detected by classifying successive segments of the audio signal, as long as microphone 222 and accelerometer 212 are operational.

FIG. 7B shows a block diagram of CNN layers for processing a plurality of audio segments and a plurality of motion segments, consistent with one or more exemplary embodiments of the present disclosure. In an exemplary embodiment, CNN layers 702 may include a fifth convolutional layer 712, a third average pooling layer 714, a sixth convolutional layer 716, a fourth average pooling layer 718, and a seventh convolutional layer 722. In an exemplary embodiment, steps 185-189 may be implemented utilizing CNN layers 702. Exemplary numerical values for different layers of CNN layers 702 for a sampling rate of 125 Hz are shown in FIG. 7B.

For further detail with respect to step 185, an exemplary first filtered cough feature set 724 may be extracted from an audio/motion segment 726 by applying fifth convolutional layer 712 on audio/motion segment 726. In an exemplary embodiment, audio/motion segment 726 may include the first audio segment and the first motion segment. An exemplary sequence folding layer 728 may be applied on a plurality of audio/motion segment 708 to extract audio/motion segment 726 from plurality of audio/motion segment 708 by splitting plurality of audio/motion segment 708. In an exemplary embodiment, plurality of audio/motion segment 708 may include plurality of audio segments 708A and a plurality of motion segments 708B. In an exemplary embodiment, fifth convolutional layer 712 may include a fifth plurality of convolution filters that may be configured to perform convolution operations on their respective inputs.

In further detail with regards to step 186, an exemplary first averaged cough feature set 730 may be generated by applying third average pooling layer 714 on first filtered cough feature set 724. In an exemplary embodiment, third average pooling layer 714 may be configured to reduce the size of first filtered cough feature set 724 by replacing different subsets of first filtered cough feature set 724 with average values of elements in the subsets.

For further detail regarding step 187, an exemplary second filtered cough feature set 732 may be generated by applying sixth convolutional layer 716 on first averaged cough feature set 730. In an exemplary embodiment, sixth convolutional layer 716 may include a sixth plurality of convolution filters that may be configured to perform convolution operations on their respective inputs.

In further detail with respect to step 188, an exemplary second averaged cough feature set 734 may be generated by applying fourth average pooling layer 718 on second filtered cough feature set 732. In an exemplary embodiment, fourth average pooling layer 718 may be configured to reduce the size of second filtered cough feature set 732 by replacing different subsets of second filtered cough feature set 732 with average values of elements in the subsets.

In further detail regarding step 189, an exemplary cough input 738 of PPG cough input sequence 706 may be generated by applying seventh convolutional layer 722 on second averaged cough feature set 734. In an exemplary embodiment, seventh convolutional layer 722 may include a seventh plurality of convolution filters that may be configured to perform convolution operations on their respective inputs. As a result, in an exemplary embodiment, cough input 738 may be obtained from audio/motion segment 726 by applying CNN layers 702 on audio/motion segment 726. Other exemplary cough inputs of cough input sequence 706 may be similarly obtained.

FIG. 7C shows a block diagram of LSTM layers for detecting a cough occurrence, consistent with one or more exemplary embodiments of the present disclosure. In an exemplary embodiment, LSTM layers 704 may include a third LSTM layer 740, a fourth LSTM layer 742, a second fully connected layer 744, and a classification layer 746. In an exemplary embodiment, steps 190-193 may be implemented utilizing LSTM layers 704. Exemplary numerical values for different layers of LSTM layers 704 for a sampling rate of 125 Hz are shown in FIG. 7C.

For further detail with respect to step 190, an exemplary third LSTM feature set 748 may be extracted from cough input sequence 706 by applying third LSTM layer 740 on cough input sequence 706. In an exemplary embodiment, cough input sequence 706 may be obtained by merging receiving each of cough inputs (for example, cough input 738) and merging the received cough inputs. An exemplary sequence unfolding layer 750 may be applied on each cough input to generate merged cough inputs 752. An exemplary flatten layer 754 may receive and convert merged cough inputs 752 to cough input sequence 706. In an exemplary embodiment, third LSTM layer 740 may include a third plurality of LSTM units.

In further detail with regards to step 191, an exemplary fourth LSTM feature set 756 may be generated from third LSTM feature set 748 by applying fourth LSTM layer 742 on third LSTM feature set 748. In an exemplary embodiment, fourth LSTM layer 742 may include a fourth plurality of LSTM units.

For further detail regarding step 192, an exemplary cough fully connected feature set 758 may be generated from fourth LSTM feature set 756 by applying second fully connected layer 744 on fourth LSTM feature set 756. In an exemplary embodiment, second fully connected layer 744 may include a number of neurons that may be configured to receive and process respective data from every neuron in fourth LSTM layer 742.

In further detail with respect to step 193, cough detection signal 710 may be obtained by applying an exemplary classification method on cough fully connected feature set 758. In an exemplary embodiment, classification layer 746 may include a number of neurons that may be configured to apply the classification method on cough fully connected feature set 758. An exemplary classification may result in two different values for cough detection signal 710 that may represent a cough event class and a non-cough event class.

In an exemplary embodiment, measuring each of the plurality of physiological parameters once per a respective time period may further include adjusting each respective time period of the plurality of time periods based on a measured value of a respective physiological parameter of the plurality of physiological parameters. For example, physiological parameters of a healthy subject may be configured to be checked few times day, whereas the parameters of a patient who is critically ill may be configured to be checked once every half an hour. In an exemplary embodiment, an initial duration may be assigned for each time period to set the measurement frequency of each of the physiological parameters under normal conditions of subject 208. In an exemplary embodiment, if the health conditions of subject 208 changes, specific physiological parameters may be measured more frequently (based on a decision of a user) by reducing respective time periods that are associated with the selected physiological parameters. As a result, in an exemplary embodiment, power consumption of system 200 may become more efficient since different parts of system 200 (such as different sensors of sensor package 202) may be configured to operate at specific time intervals for necessary measurements. Therefore, unnecessary power consumption may be avoided.

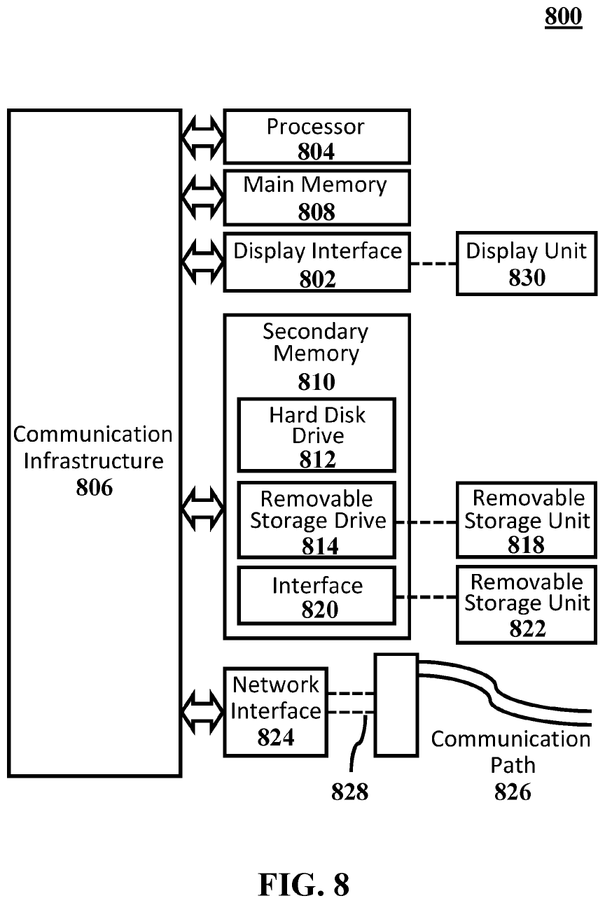

FIG. 8 shows an example computer system 800 in which an embodiment of the present invention, or portions thereof, may be implemented as computer-readable code, consistent with exemplary embodiments of the present disclosure. For example, different steps of method 100 and system 200 (such as processor 204 and processing unit 216) may be implemented in computer system 800 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-7C.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 804 may be a special purpose (e.g., a graphical processing unit) or a general-purpose processor device. As will be appreciated by persons skilled in the relevant art, processor device 804 may also be a single processor in a multi-core/multiprocessor system, such system operating alone, or in a cluster of computing devices operating in a cluster or server farm. Processor device 804 may be connected to a communication infrastructure 806, for example, a bus, message queue, network, or multi-core message-passing scheme.

In an exemplary embodiment, computer system 800 may include a display interface 802, for example a video connector, to transfer data to a display unit 830, for example, a monitor. Computer system 800 may also include a main memory 808, for example, random access memory (RAM), and may also include a secondary memory 810. Secondary memory 810 may include, for example, a hard disk drive 812, and a removable storage drive 814. Removable storage drive 814 may include a floppy disk drive, a magnetic tape drive, an optical disk drive, a flash memory, or the like. Removable storage drive 814 may read from and/or write to a removable storage unit 818 in a well-known manner. Removable storage unit 818 may include a floppy disk, a magnetic tape, an optical disk, etc., which may be read by and written to by removable storage drive 814. As will be appreciated by persons skilled in the relevant art, removable storage unit 818 may include a computer usable storage medium having stored therein computer software and/or data.

In alternative implementations, secondary memory 810 may include other similar means for allowing computer programs or other instructions to be loaded into computer system 800. Such means may include, for example, a removable storage unit 822 and an interface 820. Examples of such means may include a program cartridge and cartridge interface (such as that found in video game devices), a removable memory chip (such as an EPROM, or PROM) and associated socket, and other removable storage units 822 and interfaces 820 which allow software and data to be transferred from removable storage unit 822 to computer system 800.

Computer system 800 may also include a communications interface 824. Communications interface 824 allows software and data to be transferred between computer system 800 and external devices. Communications interface 824 may include a modem, a network interface (such as an Ethernet card), a communications port, a PCMCIA slot and card, or the like. Software and data transferred via communications interface 824 may be in the form of signals, which may be electronic, electromagnetic, optical, or other signals capable of being received by communications interface 824. These signals may be provided to communications interface 824 via a communications path 826. Communications path 826 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 818, removable storage unit 822, and a hard disk installed in hard disk drive 812. Computer program medium and computer usable medium may also refer to memories, such as main memory 808 and secondary memory 810, which may be memory semiconductors (e.g. DRAMs, etc.).

Computer programs (also called computer control logic) are stored in main memory 308 and/or secondary memory 810. Computer programs may also be received via communications interface 824. Such computer programs, when executed, enable computer system 800 to implement different embodiments of the present disclosure as discussed herein. In particular, the computer programs, when executed, enable processor device 804 to implement the processes of the present disclosure, such as the operations in method 100 illustrated by flowcharts of FIGS. 1A-1K discussed above. Accordingly, such computer programs represent controllers of computer system 800. Where exemplary embodiments of method 100 are implemented using software, the software may be stored in a computer program product and loaded into computer system 800 using removable storage drive 814, interface 820, and hard disk drive 812, or communications interface 824.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for health monitoring of a subject, the method comprising measuring each of a plurality of physiological parameters of the subject once per a respective time period of a plurality of time periods, measuring each of the plurality of physiological parameters comprising measuring a heart rate of the plurality of physiological parameters by:
installing a sensor package by placing an electrocardiography (ECG) electrodes pair of the sensor package on a region at a right side of a chest of the subject, the region comprising around the anterior edge of the right serratus anterior muscle of the subject;
placing an accelerometer of the sensor package on the region;
acquiring an ECG signal of the subject by acquiring each ECG sample of the ECG signal utilizing the ECG electrodes pair;
acquiring, utilizing the accelerometer, a motion signal of the subject by acquiring each motion sample of the motion signal simultaneously with acquiring a respective ECG sample of the ECG signal;
calculating, utilizing one or more processors, a short-time Fourier transform (STFT) of the ECG signal responsive to a magnitude of a respective motion sample of the motion signal being smaller than a predetermined motion threshold;
extracting, utilizing the one or more processors, a plurality of P waves, a plurality of QRS complexes, and a plurality of T waves from the STFT by applying a long short-term memory (LSTM) neural network on the STFT; and
estimating, utilizing the one or more processors, the heart rate by calculating a number of the plurality of QRS complexes in a given period of time.

2. The method of claim 1, wherein placing the ECG electrodes pair comprises placing a pair of biocompatible cohesive ECG electrodes one inch apart on the region in a vertical orientation.

3. The method of claim 1, wherein measuring each of the plurality of physiological parameters further comprises measuring an Oxygen saturation level (SpO₂) of the plurality of physiological parameters by:
placing a photoplethysmography (PPG) sensor of the sensor package on the region; and
measuring the SpO₂ utilizing the PPG sensor.

4. The method of claim 3, wherein measuring each of the plurality of physiological parameters further comprises estimating, utilizing the one or more processors, a respiratory rate of the plurality of physiological parameters by:
acquiring, utilizing the PPG sensor, a PPG signal from the subject by acquiring each PPG sample of the PPG signal simultaneously with acquiring a respective ECG sample of the ECG signal;
responsive to a magnitude of a respective motion sample of the motion signal being smaller than the predetermined motion threshold, extracting a respiratory component of the PPG signal by removing a cardiac component of the PPG signal utilizing a sequential harmonic infinite impulse response (IIR) notch filter based on the heart rate;
extracting a refined signal from the respiratory component by applying a band-pass filter on the respiratory component; and
obtaining the respiratory rate by applying an adaptive lattice notch filter (ALNF) on the refined signal.

5. The method of claim 4, wherein measuring each of the plurality of physiological parameters further comprises estimating, utilizing the one or more processors, a systolic blood pressure of the plurality of physiological parameters and a diastolic blood pressure of the plurality of physiological parameters by:
segmenting the PPG signal to a plurality of PPG segments by extracting each of the plurality of PPG segments from the PPG signal at a respective time interval corresponding to a respective QRS complex of the plurality of QRS complexes; and
obtaining the systolic blood pressure and the diastolic blood pressure by applying an end-to-end neural network on the plurality of PPG segments, comprising:
extracting a first filtered PPG feature set of a first plurality of filtered PPG feature sets from a PPG segment of the plurality of PPG segments by applying a first convolutional layer of the end-to-end neural network on the PPG segment, the first convolutional layer comprising a first plurality of convolution filters;
generating a first averaged PPG feature set of a first plurality of averaged PPG feature sets by applying a first average pooling layer of the end-to-end neural network on the first filtered PPG feature set;
generating a second filtered PPG feature set of a second plurality of filtered PPG feature sets by applying a second convolutional layer of the end-to-end neural network on the first averaged PPG feature set, the second convolutional layer comprising a second plurality of convolution filters;
generating a second averaged PPG feature set of a second plurality of averaged PPG feature sets by applying a second average pooling layer of the end-to-end neural network on the second filtered PPG feature set;
generating a third filtered PPG feature set of a third plurality of filtered PPG feature sets by applying a third convolutional layer of the end-to-end neural network on the second averaged PPG feature set, the third convolutional layer comprising a third plurality of convolution filters;
generating a PPG input of a PPG input sequence by applying a fourth convolutional layer of the end-to-end neural network on the third filtered PPG feature set, the fourth convolutional layer comprising a fourth plurality of convolution filters;
extracting a first LSTM feature set from the PPG input sequence by applying a first LSTM layer of the end-to-end neural network on the PPG input sequence, the first LSTM layer comprising a first plurality of LSTM units;
generating a second LSTM feature set by applying a second LSTM layer of the end-to-end neural network on the first LSTM feature set, the second LSTM layer comprising a second plurality of LSTM units;
generating a PPG fully connected feature set by applying a first fully connected layer of the end-to-end neural network on the second LSTM feature set; and
obtaining estimated values of the systolic blood pressure and the diastolic blood pressure by applying a regression method on the PPG fully connected feature set through feeding the PPG fully connected feature set to a regression layer of the end-to-end neural network.

6. The method of claim 5, wherein applying the end-to-end neural network comprises:
providing a training data set associated with the plurality of physiological parameters, the training data set comprising a plurality of reference ECG signals and a plurality of reference PPG signals;
acquiring, utilizing a cuff-based measurement method, calibration values of the systolic blood pressure and the diastolic blood pressure of the subject;
acquiring, utilizing a plurality of ECG electrodes, a standard ECG signal of the subject;
providing an updated training data set by adding the calibration values and the standard ECG signal to the training data set; and
training the end-to-end neural network utilizing the updated training data set.

7. The method of claim 6, wherein estimating the systolic blood pressure and the diastolic blood pressure further comprises removing an estimation offset of the systolic blood pressure and the diastolic blood pressure by subtracting each calibration value of the systolic blood pressure and the diastolic blood pressure from a respective estimated value of the systolic blood pressure and the diastolic blood pressure.

8. The method of claim 6, wherein installing the sensor package comprises:
moving the sensor package on the region simultaneously with acquiring the ECG signal and acquiring the PPG signal;
calculating, utilizing the one or more processors, a plurality of quality factors simultaneously with moving the sensor package, each of the plurality of quality factors associated with a respective location of a plurality of locations in the region, calculating the plurality of quality factors comprising:
measuring a first correlation between the ECG signal and a reference ECG signal of the plurality of reference ECG signals;
measuring a second cross-correlation between the PPG signal and a reference PPG signal of the plurality of reference PPG signals; and
calculating a quality factor of the plurality of quality factors by averaging the first correlation and the second correlation;
obtaining a subset of the plurality of quality factors, each respective quality factor in the subset comprising a value larger than a predetermined ratio of a largest quality factor of the plurality of quality factors;
sticking a skin attachment piece at an optimal location of the plurality of locations, the optimal location associated with a respective quality factor in the subset; and
installing the sensor package on the skin attachment piece.

9. The method of claim 1, wherein measuring each of the plurality of physiological parameters further comprises estimating a body temperature of the plurality of physiological parameters by measuring a radiation power of a thermal radiation from the subject's body utilizing a thermopile sensor of the sensor package.

10. The method of claim 1, wherein measuring each of the plurality of physiological parameters further comprises detecting a cough occurrence of the plurality of physiological parameters by:
recording, utilizing a microphone, an audio signal simultaneously with acquiring the motion signal, the audio signal associated with a motion sample of the motion signal; and
detecting, utilizing the one or more processors, the cough occurrence responsive to:
a magnitude of the motion sample being larger than a predetermined cough threshold;
a center frequency of the audio signal being located in a predetermined frequency range; and
a peak amplitude of the audio signal being larger than a predetermined amplitude threshold.

11. The method of claim 10, wherein detecting the cough occurrence comprises:
segmenting the audio signal to a plurality of audio segments by extracting each of the plurality of audio segments from the audio signal at a predefined time interval;
segmenting the motion signal to a plurality of motion segments by extracting each of the plurality of motion segments from the motion signal at the predefined time interval;
extracting a first filtered cough feature set of a first plurality of filtered cough feature sets from a first audio segment of the plurality of audio segments and a first motion segment of the plurality of motion segments by applying a fifth convolutional layer on the first audio segment and the first motion segment, the fifth convolutional layer comprising a fifth plurality of convolution filters;
generating a first averaged cough feature set of a first plurality of averaged cough feature sets by applying a third average pooling layer on the first cough feature set;
generating a second filtered cough feature set of a second plurality of filtered cough feature sets by applying a sixth convolutional layer on the first averaged cough feature set, the sixth convolutional layer comprising a sixth plurality of convolution filters;
generating a second averaged cough feature set of a second plurality of averaged cough feature sets by applying a fourth average pooling layer on the second filtered cough feature set;
generating a cough input of a cough input sequence by applying a seventh convolutional layer on the second averaged cough feature set, the seventh convolutional layer comprising a seventh plurality of convolution filters;
extracting a third LSTM feature set from the cough input sequence by applying a third LSTM layer on the cough input sequence, the third LSTM layer comprising a third plurality of LSTM units;
generating a fourth LSTM feature set by applying a fourth LSTM layer on the third LSTM feature set, the fourth LSTM layer comprising a fourth plurality of LSTM units;
generating a cough fully connected feature set by applying a second fully connected layer on the fourth LSTM feature set; and
classifying the first audio segment in one of a cough event class or a non-cough event class by applying a classification method on the cough fully connected feature set through feeding the cough fully connected feature set to a classification layer.

12. The method of claim 1, wherein measuring each of the plurality of physiological parameters once per a respective time period further comprises adjusting each respective time period of the plurality of time periods based on a measured value of a respective physiological parameter of the plurality of physiological parameters.

13. A system for health monitoring of a subject, the system comprising:
a sensor package comprising:
an electrocardiography (ECG) electrodes pair configured to:
be placed on a region at a right side of a chest of the subject, the region comprising the anterior edge of the right serratus anterior muscle of the subject; and
acquire an ECG signal of the subject by acquiring each ECG sample of the ECG signal; and
an accelerometer configured to:
be placed on the region; and
acquire a motion signal of the subject by acquiring each motion sample of the motion signal simultaneously with acquiring a respective ECG sample of the ECG signal;
a memory having processor-readable instructions stored therein; and
one or more processors configured to access the memory and execute the processor-readable instructions, which, when executed by the one or more processors configures the one or more processors to perform a method, the method comprising measuring each of a plurality of physiological parameters of the subject once per a respective time period of a plurality of time periods, measuring each of the plurality of physiological parameters comprising measuring a heart rate of the plurality of physiological parameters by:
calculating a short-time Fourier transform (STFT) of the ECG signal responsive to a magnitude of a respective motion sample of the motion signal being smaller than a predetermined motion threshold;
extracting a plurality of P waves, a plurality of QRS complexes, and a plurality of T waves from the STFT by applying a long short-term memory (LSTM) neural network on the STFT; and
estimating the heart rate by calculating a number of the plurality of QRS complexes in a given period of time.

14. The system of claim 13, wherein the ECG electrodes pair comprise a pair of biocompatible cohesive ECG electrodes configured to be placed one inch apart on the region in a vertical orientation.

15. The system of claim 13, wherein the sensor package further comprises:
a photoplethysmography (PPG) sensor configured to:
be placed on the region; and
measure an Oxygen saturation level (SpO₂) of the plurality of physiological parameters; and
a skin attachment piece configured to:
be installed to the sensor package; and
attach the sensor package at an optimal location of a plurality of locations by being stuck to the optimal location, the optimal location associated with a largest quality factor of a plurality of quality factors, each of the plurality of quality factors associated with a respective location of a plurality of locations in the region and comprising an average value of a first cross-correlation between the ECG signal and a reference ECG signal and a second cross-correlation between the PPG signal and a reference PPG signal.

16. The system of claim 15, wherein measuring each of the plurality of physiological parameters further comprises estimating a respiratory rate of the plurality of physiological parameters by:
acquiring, utilizing the PPG sensor, a PPG signal from the subject by acquiring each PPG sample of the PPG signal simultaneously with acquiring a respective ECG sample of the ECG signal;
responsive to a magnitude of a respective motion sample of the motion signal being smaller than the predetermined motion threshold, extracting a respiratory component of the PPG signal by removing a cardiac component of the PPG signal utilizing a sequential harmonic infinite impulse response (IIR) notch filter based on the heart rate;
extracting a refined signal from the respiratory component by applying a band-pass filter on the respiratory component; and
obtaining the respiratory rate by applying an adaptive lattice notch filter (ALNF) on the refined signal.

17. The system of claim 16, wherein measuring each of the plurality of physiological parameters further comprises estimating a systolic blood pressure of the plurality of physiological parameters and a diastolic blood pressure of the plurality of physiological parameters by:
segmenting the PPG signal to a plurality of PPG segments by extracting each of the plurality of PPG segments from the PPG signal at a respective time interval corresponding to a respective QRS complex of the plurality of QRS complexes;
obtaining the systolic blood pressure and the diastolic blood pressure by applying an end-to-end neural network on the plurality of PPG segments, comprising:
extracting a first filtered PPG feature set of a first plurality of filtered PPG feature sets from a PPG segment of the plurality of PPG segments by applying a first convolutional layer of the end-to-end neural network on the PPG segment, the first convolutional layer comprising a first plurality of convolution filters;
generating a first averaged PPG feature set of a first plurality of averaged PPG feature sets by applying a first average pooling layer of the end-to-end neural network on the first filtered PPG feature set;
generating a second filtered PPG feature set of a second plurality of filtered PPG feature sets by applying a second convolutional layer of the end-to-end neural network on the first averaged PPG feature set, the second convolutional layer comprising a second plurality of convolution filters;
generating a second averaged PPG feature set of a second plurality of averaged PPG feature sets by applying a second average pooling layer of the end-to-end neural network on the second filtered PPG feature set;
generating a third filtered PPG feature set of a third plurality of filtered PPG feature sets by applying a third convolutional layer of the end-to-end neural network on the second averaged PPG feature set, the third convolutional layer comprising a third plurality of convolution filters;
generating a PPG input of a PPG input sequence by applying a fourth convolutional layer of the end-to-end neural network on the third filtered PPG feature set, the fourth convolutional layer comprising a fourth plurality of convolution filters;
extracting a first LSTM feature set from the PPG input sequence by applying a first LSTM layer of the end-to-end neural network on the PPG input sequence, the first LSTM layer comprising a first plurality of LSTM units;
generating a second LSTM feature set by applying a second LSTM layer of the end-to-end neural network on the first LSTM feature set, the second LSTM layer comprising a second plurality of LSTM units;
generating a PPG fully connected feature set by applying a first fully connected layer of the end-to-end neural network on the second LSTM feature set; and
obtaining estimated values of the systolic blood pressure and the diastolic blood pressure by applying a regression method on the PPG fully connected feature set through feeding the PPG fully connected feature set to a regression layer of the end-to-end neural network.

18. The system of claim 13, wherein the sensor package further comprises:
a thermopile sensor configured to estimate a body temperature of the plurality of physiological parameters by measuring a radiation power of a thermal radiation from the subject's body; and
a microphone configured to record an audio signal simultaneously with acquiring the motion signal, the audio signal associated with a motion sample of the motion signal.

19. The system of claim 18, wherein measuring each of the plurality of physiological parameters further comprises detecting a cough occurrence of the plurality of physiological parameters by:
segmenting the audio signal to a plurality of audio segments by extracting each of the plurality of audio segments from the audio signal at a predefined time interval;
segmenting the motion signal to a plurality of motion segments by extracting each of the plurality of motion segments from the motion signal at the predefined time interval;
extracting a first filtered cough feature set of a first plurality of filtered cough feature sets from a first audio segment of the plurality of audio segments and a first motion segment of the plurality of motion segments by applying a fifth convolutional layer on the first audio segment and the first motion segment, the fifth convolutional layer comprising a fifth plurality of convolution filters;
generating a first averaged cough feature set of a first plurality of averaged cough feature sets by applying a third average pooling layer on the first cough feature set;
generating a second filtered cough feature set of a second plurality of filtered cough feature sets by applying a sixth convolutional layer on the first averaged cough feature set, the sixth convolutional layer comprising a sixth plurality of convolution filters;
generating a second averaged cough feature set of a second plurality of averaged cough feature sets by applying a fourth average pooling layer on the second filtered cough feature set;
generating a cough input of a cough input sequence by applying a seventh convolutional layer on the second averaged cough feature set, the seventh convolutional layer comprising a seventh plurality of convolution filters;
extracting a third LSTM feature set from the cough input sequence by applying a third LSTM layer on the cough input sequence, the third LSTM layer comprising a third plurality of LSTM units;
generating a fourth LSTM feature set by applying a fourth LSTM layer on the third LSTM feature set, the fourth LSTM layer comprising a fourth plurality of LSTM units;
generating a cough fully connected feature set by applying a second fully connected layer on the fourth LSTM feature set; and
classifying the first audio segment in one of a cough event class or a non-cough event class by applying a classification method on the cough fully connected feature set through feeding the cough fully connected feature set to a classification layer.

20. The system of claim 13, wherein measuring each of the plurality of physiological parameters once per a respective time period further comprises adjusting each respective time period of the plurality of time periods based on a measured value of a respective physiological parameter of the plurality of physiological parameters.
